SEQUENCE LISTING (26 sequences)

SEQ ID NO: 1 - DNA, 1786 bp, Homo sapiens
agtcgggccgcggcgacggcggcaggagcgcgtcccggcgccgcctcgggctccgctcggctcgggggctgcttcgggaggaggagagccaagggaggcgccaggcccgcgggccgggcgcatggcttagggacgctcccggccgccgcagccccagcatggggaaacttcactccaagccggccgccgtgtgcaagcgcagggagagcccggaaggtgacagcttcgccgtgagcgctgcctgggctcggaagggcatcgaggagtggatcgggagacagcgctgcccgggcggtgtctcgggaccccgacagctgcggttggcgggcaccataggccgaagcacccgggagctcgtgggcgacgtgttgagagacacgctcagcgaggaagaggaggacgactttcggctggaagtggccctgcctcctgagaagactgacgggctgggcagcggagatgagaagaagatggagagagtgagcgaaccctgcccaggctccaagaagcagctgaagtttgaagagctccagtgcgacgtgtccatggaggaggacagccggcaggagtggaccttcaccctgtatgactttgacaacaacggcaaggtcacccgagaggacatcaccagcttgctgcacaccatctatgaggtggtggactcctctgtcaaccactccccaacatccagcaagatgctgcgggtaaagctcaccgtggcccccgatggcagccagagcaagaggagcgtccttgtcaatcaggctgacctgcagagcgcaaggccccgagcagagaccaagcccactgaggacctgcggagctgggagaagaagcagcgagccccgctcaggttccagggtgacagccgcctggagcagtctggctgctaccaccattgcgtagatgagaacatcgagaggagaaaccactacttagatctcgccgggatagaaaactacacgtcccaatttgggcctggctccccttccgtggcccagaagtcagaactgcccccccgcacctccaatcccactcgatctcgctcccatgagccggaagccatccacatcccacaccgaaagccccaaggcgtggacccggcctccttccacttccttgacaccccaatcgccaaggtctcagagctccagcaacggctccggggcacccaggacgggagcaagcactttgtgaggtcccccaaggcccagggcaagagtgtgggtgtgggccacgtggccagaggggcaagaaacaagccccctctgggacccgccatccctgcggtgtccccctccgcccacctggctgccagcccggccctcctcccctccctagcccccctcgggcacaagaagcacaagcaccgagccaaggagagccagcagggctgccggggcctgcaggcaccactggcctcaggtggccctgtcctggggcgggagcacctgcgggagctgcccgccttggtggtgtatgagagccaggccgggcagccggtccagagacatgagcaccaccaccaccatgaacatcaccaccattaccaccacttctaccagacatagagcccctccccagggccccaccctgccatatgaaggaccccacccccgacaccacaaggcattattattctattaattattgttattatgatgattattgttattaataattattgttactccactaatatttagctagcctacatgtagaagatctatggaaacacagaactaaacttttatttatatgttaaaaaaaaaaaaaaaaaaaaaaa

SEQ ID NO: 2 - DNA, 1416 bp, Mus sp.
atggggaaacttcactcgaagccggccgccgtgtgcaagcgcagggagagcccggaaggtgacagctttgctgtaagcgctgcttgggcaaggaaaggcatcgaggagtggatcgggaggcagcgctgtccaggcagcgtctcaggaccccgtcagctgagattggcaggcactgttggtcgaggcactcgggaactcgtgggtgacacttctagagaggctctcggtgaggaggacgaggacgacttccccctagaagtggccctgccgcctgagaagatcgacagcctaggtagtggagatgagaagagaatggagagactgagcgaacctggccaggcctccaagaagcagctcaagtttgaagagctacagtgtgatgtctctgtggaggaggacagccggcaagagtggactttcactctatatgacttcgacaacaatggcaaagtgacccgtgaggacattaccagcttgctgcataccatctatgaagtggttgactcctctgtgaaccattcccccacatcaagcaagacactgcgggtgaagctcaccgtggctcctgacgggagccagagtaagaggagcgtccttttcaaccataccgatctgcagagcacaaggccccgagcagacaccaaacccgctgaggagctgcgtggctgggagaagaagcagcgagccccactcaggttccagggtgacagccacctggagcagccagactgctaccaccattgcgtggatgagaacattgagaggagaaaccactacctagacctggcggggatagagaactacacgtctcagtttggaccgggatccccttcggtggcccagaagtcagagctgccccctcgaatctccaaccccactcgctctcgctcccacgagccagaagctgcccacatcccacaccggaggccccaaggtgtggacccaggctccttccacctccttgacaccccatttgccaaggcatcagagctccagcaacggctccggggcactcaggatgggagcaagcactttgtgaggtcccccaaggcccagggcaagaacatgggtatgggccacggggccagaggtgcaagaagcaagcctccactggtacccaccacccatactgtctccccctctgcccatctggccaccagcccagcccttctccccaccctggcacccctggggcacaagaaacacaagcatcgagccaaggagagccaggcgagctgccggggcctgcagggccccctggctgcaggaggctccaccgtcatggggcgggagcaggtgagggagctgcctgccgtggtggtgtacgagagccaggctgggcaggccgtccagagacacgaacaccatcaccaccaccaacatcaccaccattatcaccacttctatcagccctag

SEQ ID NO: 3 - protein, 470 aa, Homo sapiens
MGKLHSKPAAVCKRRESPEGDSFAVSAAWARKGIEEWIGRQRCPGGVSGPRQLRLAGTIGRSTRELVGDVLRDTLSEEEEDDFRLEVALPPEKTDGLGSGDEKKMERVSEPCPGSKKQLKFEELQCDVSMEEDSRQEWTFTLYDFDNNGKVTREDITSLLHTIYEVVDSSVNHSPTSSKMLRVKLTVAPDGSQSKRSVLVNQADLQSARPRAETKPTEDLRSWEKKQRAPLRFQGDSRLEQSGCYHHCVDENIERRNHYLDLAGIENYTSQFGPGSPSVAQKSELPPRTSNPTRSRSHEPEAIHIPHRKPQGVDPASFHFLDTPIAKVSELQQRLRGTQDGSKHFVRSPKAQGKSVGVGHVARGARNKPPLGPAIPAVSPSAHLAASPALLPSLAPLGHKKHKHRAKESQQGCRGLQAPLASGGPVLGREHLRELPALVVYESQAGQPVQRHEHHHHHEHHHHYHHFYQT

SEQ ID NO: 4 - protein, 471 aa, Mus sp.
MGKLHSKPAAVCKRRESPEGDSFAVSAAWARKGIEEWIGRQRCPGSVSGPRQLRLAGTVGRGTRELVGDTSREALGEEDEDDFPLEVALPPEKIDSLGSGDEKRMERLSEPGQASKKQLKFEELQCDVSVEEDSRQEWTFTLYDFDNNGKVTREDITSLLHTIYEVVDSSVNHSPTSSKTLRVKLTVAPGDSQSKRSVLFNHTDLQSTRPRADTKPAEELRGWEKKQRAPLRFQGDSHLEQPDCYHHCVDENIERRNHYLDLAGIENYTSQFGPGSPSVAQKSELPPRISNPTRSRSHEPEAAHIPHRRPQGVDPGSFHLLDTPFAKASELQQRLRGTQDGSKHFVRSPKAQGKNMGMGHGARGARSKPPLVPTTHTVSPSAHLATSPALLPTLAPLGHKKHKHRAKESQASCRGLQGPLAAGGSTVMGREQVRELPAVVVYESQAGQAVQRHEHHHHHEHHHHYHHFYQP

SEQ ID NO: 5 - DNA, 1859 bp, Homo sapiens
gaattcgcccttctaatacgactcactatagggcaagcagtggtaacaacgcagagtacgcggggagtcgggccgcggcgacggcggcaggagcgcgtcccggcgccgcctcgggctccgctcggctcgggggctgcttcgggaggaggagagccaagggaggcgccaggcccgcgggccgggcgcatggcttagggacgctcccggccgccgcagccccagcatggggaaacttcactccaagccggccgccgtgtgcaagcgcagggagagcccggaaggtgacagcttcgccgtgagcgctgcctgggctcggaagggcatcgaggagtggatcgggagacagcgctgcccgggcggtgtctcgggaccccgacagctgcggttggcgggcaccataggccgaagcacccgggagctcgtgggcgacgtgttgagagacacgctcagcgaggaagaggaggacgactttcggctggaagtggccctgcctcctgagaagactgacgggctgggcagcggagatgagaagaagatggagagagtgagcgaaccctgcccaggctccaagaagcagctgaagtttgaagagctccagtgcgacgtgtccatggaggaggacagccggcaggagtggaccttcaccctgtatgactttgacaacaacggcaaggtcacccgagaggacatcaccagcttgctgcacaccatctatgaggtggtggactcctctgtcaaccactccccaacatccagcaagatgctgcgggtaaagctcaccgtggcccccgatggcagccagagcaagaggagcgtccttgtcaatcaggctgacctgcagagcgcaaggccccgagcagagaccaagcccactgaggacctgcggagctgggagaagaagcagcgagccccgctcaggttccagggtgacagccgcctggagcagtctggctgctaccaccattgcgtagatgagaacatcgagaggagaaaccactacttagatctcgccgggatagaaaactacacgtcccaatttgggcctggctccccttccgtggcccagaagtcagaactgcccccccgcacctccaatcccactcgatctcgctcccatgagccggaagccatccacatcccacaccgaaagccccaaggcgtggacccggcctccttccacttccttgacaccccaatcgccaaggtctcagagctccagcaacggctccggggcacccaggacgggagcaagcactttgtgaggtcccccaaggcccagggcaagagtgtgggtgtgggccacgtggccagaggggcaagaaacaagccccctctgggacccgccatccctgcggtgtccccctccgcccacctggctgccagcccggccctcctcccctccctagcccccctcgggcacaagaagcacaagcaccgagccaaggagagccagcagggctgccggggcctgcaggcaccactggcctcaggtggccctgtcctggggcgggagcacctgcgggagctgcccgccttggtggtgtatgagagccaggccgggcagccggtccagagacatgagcaccaccaccaccatgaacatcaccaccattaccaccacttctaccagacatagagcccctccccagggccccaccctgccatatgaaggaccccacccccgacaccacaaggcattattattctattaattattgttattatgatgattattgttattaataattattgttactccactaatatttagctagcctacatgtagaagatctatggaaacacagaactaaacttttatttatatgttaaaaaaaaaaaaaaaaaaaaaaagcggccgc

SEQ ID NO: 6 - DNA, 1401 bp, Mus sp.
atggggaaacttcactcgaagccggccgccgtgtgcaagcgcagggagagcccggaaggtgacagctttgctgtaagcgctgcttgggcaaggaaaggcatcgaggagtggatcgggaggcagcgctgtccaggcagcgtctcaggaccccgtcagctgagattggcaggcactgttggtcgaggcactcgggaactcgtgggtgacacttctagagaggctctcggtgaggaggacgaggacgacttccccctagaagtggccctgccgcctgagaagatcgacagcctaggtagtggagatgagaagagaatggagagactgagcgaacctggccaggcctccaagaagcagctcaagtttgaagagctacagtgtgatgtctctgtggaggaggacagccggcaagagtggactttcactctatatgacttcgacaacaatggcaaagtgacccgtgaggacattaccagcttgctgcataccatctatgaagtggttgactcctctgtgaaccattcccccacatcaagcaagacactgcgggtgaagctcaccgtggctcctgacgggagccagagtaagaggagcgtccttttcaaccataccgatctgcagagcacaaggccccgagcagacaccaaacccgctgaggagctgcgtggctgggagaagaagcagcgagccccactcaggttccagggtgacagccacctggagcagccagactgctaccaccattgcgtggatgagaacattgagaggagaaaccactacctagacctggcggggatagagaactacacgtctcagtttggaccgggatccccttcggtggcccagaagtcagagctgccccctcgaatctccaaccccactcgctctcgctcccacgagccagaagctgcccacatcccacaccggaggccccaaggtgtggacccaggctccttccacctccttgacaccccatttgccaaggcatcagagctccagcaacggctccggggcactcaggatgggagcaagcactttgtgaggtcccccaaggcccagggcaagaacatgggtatgggccacggggccagaggtgcaagaagcaagcctccactggtacccaccacccatactgtctccccctctgcccatctggccaccagcccagcccttctccccaccctggcacccctggggcacaagaaacacaagcatcgagccaaggagagccaggcgagctgccggggcctgcagggccccctggctgcaggaggctccaccgtcatggggcgggagcaggtgagggagctgcctgccgtggtggtgtacgagagccaggctgggcaggccgtccagagacacgaacaccatcaccaccaccaacatcaccaccattatcaccac

SEQ ID NO: 7 - protein, 470 aa, Homo sapiens; identical to SEQ ID NO: 3

SEQ ID NO: 8 - protein, 471 aa, Mus sp.; identical to SEQ ID NO: 4

SEQ ID NO: 9 - DNA, 6455 bp, Homo sapiens
gaattcatatgcacattaaattccagggagccctcctctaggctatttgaccctagctcaagaaagggggattaagagtcttacagggagggatccaaggtcagcatatacagttagtcagggaccagtctgtctgtgtctctctctccatggggtttactatcattgctttcccctaatggttcttactcctgcttcttcctgcttatttttcagcccacagcgaccccaagaagctgctccaacccctgggactatggagctctacagctgtagagaccaccaggaagtggactgcaggcccctggcctctccattcagattctgcaaagagatcctgatgggttgggccaatgggtcaggcatccagtcagctctggctaaggggtgaaggagtcaggtgttaccaacgtggtggcaggggccaccttgaagctgtgttctgtgccatggaagaaggaagaggaggaggaagctaagctggaagggaaggcaggtgatacaggaaaattaactatgagctttgctatagtgaccacttttccttcactccttgagctgtggccttaagaactgtgtaccaatgggaggcacttgcatagtaagtgttcatttgctgaatacttacagagggctataagtggacaaatatgtccaaaaacacatgaaacacacaccatcaacacttgcagatggtctccttcagggaacctttccacactggctctcccctcactgagctttccttcctatcaccctcccagtctaggctcctggagtcagtagttggaatctcagatgggaagaaaccttaaaagtcatctggtccagtattttccaaagcatgttccatgaacttgttttccagaaatggtttcctggtctggtgagtttaagaaaccctgcttatgacgatgctctccatttagagaatcacaaagcttggctactcaatgaaagctctgacaagtcctgcaggaaaaaacttgtcttcttttggctaagctagggctgcccaaagtttctcatggagtcctttcttgcacataataatagcatctcacaaaccagtggtcgggggaacccattacgggaaatgctaatcttctggacccttccttctattttataggtggagaggctgtgtggtggtctggttggctgcatgtaactgaaaaacaaaggcttaaaaagataggggcttcttttgctcttttgttaacaaagtctgggaatagtcaaagactggtactgtgactagaaaggcttctgatatggtttggctctgtgttcccacccaaatctcaccttgagttgtaatgatccccatatgtcaagggcaggatcaggtggaggtaattgaatcatgagggcagttaatcccatgctgttcttgtgatagtgagttctcacaggatctgatggttttataaggggcttttcccctttgctcggcacctctctctcctgctgctatgtgaagagggacgtgtttgcttctccttctgtcatgattgtaagtttcctgaggcctccccagccatgtagaactgtgagtcaattaaacctctttcctttataaattgcccagttccggtatgttcttgtagcagcctgagaacggactaatatagcttctctgcccagtgtgaagaagagcagagaggcagggctgggaggagaacaaggcacctgccaaggagatggggaggctgggctggctttccctctcctccaggctcacctgggaagcctgtgctctaaacttgctcaaacatcctgaacccaggaggagttggtggtacacaaattcaattcaattcaaccccacatccagactgtactcaagcagcagtcttttggcccagtcatctccaacctcatcttctcccctctactcccaaaccatgctctctctgctccagagccaggggcctctttgctgtttccaaaacatccatggcagtctccacttcagggcctttacatgtgctgttccctctgcctttagtacccaaacagaatggcttggagaccccagccctagttcttggggaagcccagcctcctccatctcatatctaaggcctgaggcctcctggctgcctctggctcccatcttttctcctgcagggtatctccactgtgaagattgctgttggccccattaattacctgtaggagtcatctttctgattccttaattttgtcctgtgccactaacccaggaagtggctaggattttgctgagggcttggagtaacagaggaagaagagagcactgccaatcgcatgttagagctcactgtcccaaaagtgaattggccagtccccaccttgctggctgtgccttcttaggccccattatcactctcttctcatgctgtttcctttgagatctttgttttcccttccctccaaaatgcctgatattttctcaggcagagtagtctaactttcccctcctcacccaaactaggcttccaggccctttagcaatgccaaaactcaccagggaactataaaataaagcaactccagggatccaggaggaaccagtggagacttgggaactgattttctccttcatatatccaagatcgtattattggcagtactccctccttattgaccacaacatgctcctggctgaggctgggcagagagagtttgtctccttccacagcaggtgtgatggcacctgctataggcagtgctgtgttcggcattgcattcagagatgcaagtaaggcaagaaccctggcctcaaggagccctggctccaggagtgaaacttagcctcatgcataaaataagtggagatgggagggggcagggaaaccttggctaatcagagcagagaagagcccttcaggttggaaggtcaaggagggcttcctttaggaaatggcatttgaaggggccagatcagatggcttaacttcggggacaagctttggagcagctaacttgggtgatgtaggattttttttttttttaaatctcccagctctatgtctgacagatttcacctaaaccagccttgttaaatctcaagccccatgaaacccgtttctgttataatctctctctcttatctcttccttgccttcctttcccattctcctcccccaaaggataggaaatcttcaaagaaaaagatgtgtcactgcaagtatacagcccaagaaatgggccagataaattattaaaacacacgaaaagacagtgagttatggggtgggaagccctcggaggccgaatggccaccccaggtagacagcatgctggtggcccctggagacccccttctcagagacctggacagactaacattttgccacaaggcccatctcttgggttctcaccccagatctgggtaagggtatcatgattccaatagcagtgaagtcccaggcgcctgctgggctgggaggcaccagggtgaaggtggaggggggctttgtgtctgggctggacatttgggatttactccccgcaagactcaaactgctaactgcagttttagttcttgcttttacctctttttgaactgtaaaaagaaattcccaaggggaagaggggatacttttttctcatggaagaagaaagccaggaccggtttaagaaagtaaccaactttctaagcactgtgagaaaggatgctccaagttttgctttgatttagaggcaccctggtaccagcagggaggggtgagaaaggcaaacaggaattccagacgaattccattgccttttgaggggtctgaaagagggtgcccactccgactcagatgctcaaacccctggctccctcttacacctgacccccgccgttctgccccactttttcatgttcctacagctcaggggttcttacttcagcattacccacatttgatgctggatcatttgttctggtagggtgggggggctgccttatgcattgtggtatgtgtagcagcaaccctggcctctacccactagatacctccaggcattaccaagtgtcccctagagggcaaaattgtttgctgtcaggtccttatgggatggaaagaaagaaaaatggcctgttacccctggtgtaacttactacactgtttactaattcatcatttattgtttcttgcctatcttccccctaggtgagtgggagttcgatgagagtggcagttgtctattttgttcaccgatgtatcttaggtgactaaaacaatggttgtcacatggctggcccttcatatttgtttccagatggaagactctctttctagtggtggaacattagttttgcactgtgttgggacaacctgatgtagtgaaaacaagcctgggcaatgaaatcaacagattggagttcagttcctaattgggtcatggatgaactttgtgaccttgggcaagtgagttcacctctctgagttgaataggttccctcctttctagaacaagtatgagtctgcatcagagagtggttgcgagggctacacatgatggaggatgaggactggcacatcagaagtactgaatgaagaattgtaacataaaaatgacaacagtaatatatttttgtggtttcagcactcttcaaatgaaaccacctggccaacaggattttagtgtacctgcttataacattagccttcgtttccaccaaaaagggtgttaaaaaaggaagcttggaacatgaaagtaagacacttggatgaagagatttatgactctggggggctgtgaattcctaatgtccttttgagacatgtagatcttccagagcgatgctgcccaatgcagtagccactagccaagtgcaaatggtcacttgcaatatggctagtctttgagatgtgttttaagtgtaaaatacacactgaattttaaagacttagcgcaatacaaagaatgtaaaatatctcattatatcttgaaattatactattttggatatatggtgttcccttggtgtctttggggactggttccaggatcctagaggatacccaaatccccagatgtcaagtccgctatataaaatgtcctgtagtatttgcatataacctacacacatccttctgaatactttaaatcatctctagattccttgtaattcctaatacaatgtaaatgttatgtaaatagttgttatactatattaaaaagttttttattctttatttttgctgtattattctttttgcatattttcagtccacagatggttgatgccacagatgtggaacctgtgaataaggagggctgactgtattgagttaagcgaaatatattattaatatttcatctatttctttttacttctaaaagatgtggcgacaagaaaatttaaaattacaaatgtggcccacattatatttctattgggcagtgctgctctagagagtcggcaaaaagggcagaatggagcctccattatacagatcacaaaactgagcacaggtaattcactccaaaggtcggggctggtctcactctgagctgcgggttttcttttcccacgccagagctgcctggtgccaggacgagcgtaacacggacccacagtgtccccagaagggggcaggcgttctgagagccacaaaggtggggtggaatcccttgatgtcgaccgccaccatccccctccccccgcgcgacctccccgcagagacctccccagacaaaacaaacaaacccttgggtctggcgaactgcagcggggagcggaaaccaaggaagatcaaagactcagcggttacccccttccgggccgcgcagtttggcagcgcgccccgacccgggcgggcacccacgggcccccggacgaggagatcccagagactggctgataacggggcgctttggacatttgtcgctgcctggagagggctgggctcacactggcccggggtgcgctgggggctcctcctggactccccaaataagaaactagaggagtgcggtggtggggggcgggtcacggggcgggtaatgaacactttctgcagaaggtaggtcgtgggaagactgggaaaaggcagcgctgccgaagcttgcacctgagcagctaaggtctccgctcccgacctcagtttccccacctgtaaattggagccgccgagtcccgccctgcccgtttagagagaacgtggagcggagggaagtgacagtacagttagcgatggccgggctgttctgtcccaatacgcctcctggacaagccgccccgccgggtcgccagccctggagctcggcccccggccccagaccgcggcagggagcgcggactgtgtcccgcccctcccgtcagcgccccgccctcgtccccgcccatgccccgcctccggccccgcccccgccgcaaccagccttgcctttgatgcgccgcaccggccaatgggcgcgcggggaggcgcgggccgcggcggcgggctgggggctcggcgctcccgggcgtc

SEQ ID NO: 10 - DNA, 120 bp, Homo sapiens
agtcgggccgcggcgacggcggcaggagcgcgtcccggcgccgcctcgggctccgctcggctcgggggctgcttcgggaggaggagagccaagggaggcgccaggcccgcgggccgggcg

SEQ ID NO: 11 - DNA, 38 bp, Homo sapiens
catggcttagggacgctcccggccgccgcagccccagc

SEQ ID NO: 12 - DNA, 24 bp, Homo sapiens
atggggaaacttcactccaagccg

SEQ ID NO: 13 - DNA, 34 bp, Homo sapiens
gccgccgtgtgcaagcgcagggagagcccggaag

SEQ ID NO: 14 - DNA, 134 bp, Homo sapiens
gtgacagcttcgccgtgagcgctgcctgggctcggaagggcatcgaggagtggatcgggagacagcgctgcccgggcggtgtctcgggaccccgacagctgcggttggcgggcaccataggccgaagcacccgg

SEQ ID NO: 15 - DNA, 67 bp, Homo sapiens
gagctcgtgggcgacgtgttgagagacacgctcagcgaggaagaggaggacgactttcggctggaag

SEQ ID NO: 16 - DNA, 107 bp, Homo sapiens
tggccctgcctcctgagaagactgacgggctgggcagcggagatgagaagaagatggagagagtgagcgaaccctgcccaggctccaagaagcagctgaagtttgaa

SEQ ID NO: 17 - DNA, 96 bp, Homo sapiens
gagctccagtgcgacgtgtccatggaggaggacagccggcaggagtggaccttcaccctgtatgactttgacaacaacggcaaggtcacccgagag

SEQ ID NO: 18 - DNA, 148 bp, Homo sapiens
gacatcaccagcttgctgcacaccatctatgaggtggtggactcctctgtcaaccactccccaacatccagcaagatgctgcgggtaaagctcaccgtggcccccgatggcagccagagcaagaggagcgtccttgtcaatcaggctg

SEQ ID NO: 19 - DNA, 85 bp, Homo sapiens
acctgcagagcgcaaggccccgagcagagaccaagcccactgaggacctgcggagctgggagaagaagcagcgagccccgctcag

SEQ ID NO: 20 - DNA, 128 bp, Homo sapiens
gttccagggtgacagccgcctggagcagtctggctgctaccaccattgcgtagatgagaacatcgagaggagaaaccactacttagatctcgccgggatagaaaactacacgtcccaatttgggcctg

SEQ ID NO: 21 - DNA, 590 bp, Homo sapiens
gctccccttccgtggcccagaagtcagaactgcccccccgcacctccaatcccactcgatctcgctcccatgagccggaagccatccacatcccacaccgaaagccccaaggcgtggacccggcctccttccacttccttgacaccccaatcgccaaggtctcagagctccagcaacggctccggggcacccaggacgggagcaagcactttgtgaggtcccccaaggcccagggcaagagtgtgggtgtgggccacgtggccagaggggcaagaaacaagccccctctgggacccgccatccctgcggtgtccccctccgcccacctggctgccagcccggccctcctcccctccctagcccccctcgggcacaagaagcacaagcaccgagccaaggagagccagcagggctgccggggcctgcaggcaccactggcctcaggtggccctgtcctggggcgggagcacctgcgggagctgcccgccttggtggtgtatgagagccaggccgggcagccggtccagagacatgagcaccaccaccaccatgaacatcaccaccattaccaccacttctaccagacatag

SEQ ID NO: 22 - DNA, 215 bp, Homo sapiens
agcccctccccagggccccaccctgccatatgaaggaccccacccccgacaccacaaggcattattattctattaattattgttattatgatgattattgttattaataattattgttactccactaatatttagctagcctacatgtagaagatctatggaaacacagaactaaacttttatttatatgttaaaaaaaaaaaaaaaaaaaaaaa

SEQ ID NO: 23 - DNA, 21 bp, Artificial Sequence
  Description of Artificial Sequence Primer
cttgccgttgttgtcaaagtc

SEQ ID NO: 24 - DNA, 20 bp, Artificial Sequence
  Description of Artificial Sequence Primer
cccagcatggggaaacttca

SEQ ID NO: 25 - DNA, 25 bp, Artificial Sequence
  Description of Artificial Sequence Antisense oligonucleotide
actcagcttggttagtgtgtcaggc

SEQ ID NO: 26 - DNA, 25 bp, Artificial Sequence
  Description of Artificial Sequence Reverse control oligonucleotide
cggactgtgtgattggttcgactca

(19) United States
(12) Patent Application Publication
(10) Pub. No.: US 2003/0186232 A1
(43) Pub. Date: Oct. 2, 2003

(54) Human and non-human primate homologues of Nkd protein, nucleic acid sequences encoding, and uses thereof

(75) Inventors: Michael Rohan, San Francisco, CA (US); Vivien W. Chan, San Francisco, CA (US); Dong Yan, Lafayette, CA (US)

Correspondence Address: Chiron Corporation, Intellectual Property, P.O. Box 8097, Emeryville, CA, 94662-8097, US

(73) Assignee: Chiron Corporation, Emeryville, CA (US)

(21) Appl. No.: 09/993,966

(22) Filed: Nov. 27, 2001

Related U.S. Application Data
(60) Provisional application No. 60/252,884, filed on Nov. 27, 2000.
(60) Provisional application No. 60/291,109, filed on May 16, 2001.
(60) Provisional application No. 60/325,571, filed on Oct. 1, 2001.

(51) Int. Cl. C12Q 1/68; C07H 21/04; A61K 38/17; C12P 21/02; C12N 5/06; C07K 14/705; C07K 16/30

(52) U.S. Cl. 435/6; 435/69.1; 435/320.1; 435/325; 530/350; 530/388.22; 536/23.5; 514/12

(57) ABSTRACT

A novel protein and corresponding DNA involved in Wnt signaling is taught. Antibodies, antisense and ribozymes that target expression of this protein are also provided. Therapies and detection methods involve detecting and regulating expression of this protein are further provided.

### CROSS-REFERENCE TO RELATED APPLICATION

This application claims priority from U.S. Provisional Serial No. 60/252,884, filed Nov. 27, 2000, and U.S. Provisional Serial No. 60/291,109, filed May 16, 2001, and U.S. Provisional Serial No. 60/325,571, filed Oct. 1, 2001, the entirety of which are incorporated herein by reference.

### FIELD OF THE INVENTION

The present invention relates to a protein and corresponding nucleic acid sequence encoding a human or a non-human primate protein that is a regulator of Wnt signaling pathways, to fragments or variants of this protein, and to methods of using this nucleic acid sequence or protein for therapy or diagnostic applications.

### BACKGROUND OF THE INVENTION

A Drosophila gene referred to as Dishevelled (Dsh) encodes an intracellular modular protein which is a component in a chain of proteins that carries the wingless signal from the cell membrane to the nucleus. Dsh is expressed in other vertebrates and possesses a well conserved structure. [Miller et al., Oncogene 18:7860 (1999); Aelrod et al., Genes Dev. 12:2160 (1998); Boutros et al., Cell 94:109 (1998); Li et al., EMBO J. 18:4233 (1999)]. All Dsh proteins studied to date have three highly conserved domains. Of these, the N-terminal DIX domain is also present in Axin, a negative regulator of wingless signaling. The internal PDZ domain has been shown to be a protein-protein interactive domain. Further, the C-terminal DEP domain has been implicated in G-protein signaling [Axelrod et al., Genes Dev. 12:2160 (1998); Boutrus et al., Cell 94:109 (1998); and Li et al., EMBO J. 18:4233 (1999)].

In addition to its being instrumental in the wingless pathway, Dsh is also essential in the planar polarity pathway in Drosophila, where it is activates Jun N-terminal Kinase (JNK). Several lines of evidence to date suggest that Dsh is differentially recruited into these two different pathways. A third known function of Dsh is that it interacts with Notch, possibly by blocking Notch signaling.

Wg/Wnt ligands and their receptors frizzled are involved in at least two pathways. One pathway is via the beta-catenin route, which exerts effects on cell growth, development and oncogenesis. It has been reported that several components of this pathway, beta-catenin, APC, axin and some Wnt ligands and receptor are mutated or mis-expressed in a variety of human cancers suggesting that this pathway may be involved in tumorigenesis [Smalley and Dale, Canc. Metast Rev. 18(2):215-230 (1999)]. The other pathway goes through Rho and c-jun N-terminal Kinase to establish planar polarity in epidermal structures. This pathway controls PCP in Drosophila and convergent extension movements [Shulman et al., Trends Genet. 14:452 (1998); Tada and Smith, Devel. 12:2227 (2000); Wallingford et al., Nature 405:81 (2000)]. Dishevelled is a proximal downstream component that is required in both pathways.

As noted, while different frizzled receptors have variable intrinsic signaling abilities, and are involved in at least the two identified different pathways, both utilize Dsh as a transduction component. Thus, two structurally related receptors signal through a common protein yet via distinct effector pathways. [Boutros and Mlodzik et al., Science 288: 1825-1828 (2000)]. For example, it has been shown that the frizzled receptors Fz1 and Fz2 both efficiently recruit Dsh to the membrane, suggesting that differential subcellular Dsh localization does not determine signaling efficiency and specificity [Boutros et al. (2000)(Id.)]. Boutros et al. also reported therein that Fz2 is a strong activator of Wnt-β-cat signaling and that Fz1 is a strong activator of the planar polarity pathway, but that both receptors possess the intrinsic potential to cross-activate either pathway [Boutros et al. (2000)(Id.)].

Although the exact function of Dishevelled in higher organisms remains to be elucidated, a strain of mice with mouse Dishevelled 1 (mDvl 1) deficiency exhibits characteristics of some neurological disorders in humans [Lijam et al., Cell 90: 895-905 (1997)]. This strain of mouse also provides a model for further study of the roles of the Dsh gene in mice.

It is known that loss of Dsh function disrupts both the canonical Wnt and PCP pathways, and also that overexpression of Dsh activates the canonical Wnt pathway but still along the PCP pathway. These results indicate that the PCP pathway is dose sensitive and can be disrupted by either over expression or under expression of the pathway [Axelrod et al., Genes Dev. 12:261 (1998); Boutros et al., Cell 94:109 (1998); Boutros and Mlodzik, Science 288:1825-1828 (2000); Wallingford et al. Nature 405:81 (2000)]. However, further understanding of the functions of Dsh in the wingless, JNK and Notch pathways will be expedited by the discovery of proteins that are physically or functionally related to Dsh, especially human proteins.

The identification of such proteins, especially human homologues, would be beneficial because, as discussed, Wnt signaling may play a role in the initiation of a number of different malignancies and other diseases including by way of example colon cancer, melanoma, hepatocellular carcinoma, breast cancer, ovarian cancer, endometrial cancer, skin cancer, polycystic kidney disease, medulloblastoma pilomatricomas, head and neck cancer, and prostate cancer. (Morin P J Bioessays, 21 (12): 1021-1030 (1999); Polakis P, Genes Dev. 14 (15): 1837-1851 (2000); Peifer M and Polakis P, Science 287:1606-9 (2000); Smalley M J and Dale T C, Cancer and Metastasis Rev. 18 (2): 215-230 (1999)). Also, a possible role of Wnt signaling in Alzheimer's disease has been suggested. [DeFerrari et al., Brain Res. Brain Res. Rev. 33(1): 1-12 (2000).] Therefore, the identification of novel proteins and corresponding genes that play an active role in Wnt signaling, and particularly those which inhibit Wnt signaling would be beneficial as these proteins may possess potential in the design of novel therapies and methods for diagnosis of cancers involving Wnt signaling. Still further, these proteins and DNA's may be useful in the identification of novel ligands and molecules that may have utility in diagnosis and treatment of diseases involving Wnt signaling.

Moreover, the identification of human genes and proteins involved in Wnt signaling may have diagnostic applications, as aberrant expression of this protein or the expression of aberrant forms thereof may correlate to the onset of diseases involving the Wnt pathway, e.g. cancers such as colon cancer.

Recently a protein involved in Wg/Wnt signaling pathway, Nkd, was identified in Drosophila (Zeng et al., Nature 403 (6771): 789-795 (2000) and in mice [Yan et al., “Mammalian Nkd is a Dishevelled Associated Regulate of Wnt Signaling Pathways”, (2000) (incorporated by reference in its entirety therein)]. Particularly, Zeng et al. (2000)(Id.) reported the identification of a Drosophila Nkd gene, which encodes a Wg-inducible inhibitor of Wg signaling that antagonizes the Wg/Wnt pathway [Zeng et al. (2000)]. Moreover, Yan et al. (2000)(Id.) describes the identification of a mouse homologue of Drosophila Nkd, and further report some of its biological effects, especially on the Wg/Wnt pathway. However, prior to the present invention, a human or non-human homologue of Nkd has never been identified. The identification of such a human or non-human homologue would be beneficial, especially given the potential application of such protein or its corresponding DNA in the design of potential therapeutics or diagnostics for cancer, e.g., breast, lung and colon cancer; and for neurological disorders such as Alzheimer's disease. More particularly, it is anticipated that detection of aberrant expression of the human Nkd protein or nucleic acid sequence transcript may provide a means of diagnosing different cancers or neurological disorders. Also, administration of the hNkd protein or analysis may be useful in the treatment of some cancers or neurological disorders that involve Wnt signaling.

### SUMMARY AND OBJECTS OF THE INVENTION

Toward that end, it is an object of the invention to provide a novel human or non-human primate Nkd protein, which associates with the Dishevelled protein (Dsh), that is involved in Wnt signaling. It is another object of the invention to provide variants of said novel human or non-human primate Nkd protein, which retain at least one biological activity of the corresponding human or non-human primate Nkd.

It is another object of the invention to provide fragments of said novel human or non-human primate Nkd protein, which retain at least one biological activity of the corresponding human or non-human primate Nkd.

It is yet another object of the invention to provide nucleic acid sequences or molecules that encode human or non-human primate Nkd, or fragments or variants of human or non-human primate Nkd which retain at least one biological activity of the corresponding human or non-human primate Nkd.

It is still another object of the invention to provide polyclonal or monoclonal antibodies that specifically bind human or non-human primate Nkd, with the proviso that such antibodies do not appreciably bind Drosophila or mouse Nkd.

It is still another object of the invention to provide antisense nucleic acid sequences or ribozymes which target human or non-human primate Nkd mRNA's in physiological or intracellular environments and which inhibit or reduce the translation thereof.

It is another object of the invention to provide nucleic acid probes which are complementary to nucleic acid sequences encoding human or non-human primate Nkd, particularly those probes that specifically bind to and form a stable duplex with human and non-human primate Nkd coding sequences and not those of Drosophila or mouse.

It is yet another object of the invention to provide agonists or antagonists of human or non-human primate Nkd.

It is another object of the invention to provide a complex comprising Dsh or a fragment or variant thereof, in association with at least and at least one human or non-human primate Nkd or a variant or a fragment thereof which retains at least one biological activity of the corresponding native human or non-human primate Nkd.

It is still another object of the invention to utilize human or non-human primate Nkd, or a variant or fragment thereof to activate the JNK pathway.

It is another object of the invention to inhibit Wnt signaling in a mammalian cell, preferably a human or non-human primate cell, by overexpressing therein human or non-human primate Nkd, or a variant or fragment thereof, which possesses at least one biological activity of native human or non-human primate Nkd.

It is still another object of the invention to provide human or non-human cells wherein the expression of one or both alleles of human or non-human Nkd has been knocked-out.

It is yet another object of the invention to provide a novel method of treating a disease, in particular a cancer, neurological disorder or other disease which involves aberrant Wnt signaling, by the administration of an amount of human or non-human Nkd or a variant or fragment thereof, that retains at least one biological activity of native human or non-human primate Nkd, effective to inhibit Wnt signaling.

It is yet another object of the invention to treat a cancer which involves Wnt signaling, e.g., breast or lung cancer by administering an effective amount of an agonist of human or non-human primate Nkd.

It is a more specific object of the invention to provide an isolated nucleic acid molecule that comprises the nucleic acid sequence contained in SEQ ID NO:1, or a fragment of the coding sequence which is greater than 50 nucleotides in length; or a nucleic acid molecule that is at least 90% identical to the nucleic acid molecule contained in SEQ ID NO:1 which encodes a protein possessing at least one biological activity of the protein encoded by SEQ ID NO:5.

It is still more another object of the invention to utilize the regulatory regions of the hNkd gene, especially the promoter, to regulate the expression of coding sequences other than hNkd.

It is also an object of the invention to utilize the hNkd promoter sequence or a fragment thereof comprising at least so contiguous nucleotides to identify other promoters, e.g. others that are responsive to beta-catenin or other molecules involved in Wnt signaling.

It is another object of the invention to provide a nucleic acid sequence that encodes the protein having the amino acid sequence contained in SEQIDNO:7, or a nucleic acid that encodes a polypeptide that is at least 90% identical to the polypeptide having the amino acid sequence contained in SEQ ID NO:7, or a nucleic acid sequence that encodes a fragment of the polypeptide having the amino acid sequence contained in SEQ ID NO:7 wherein said fragment is at least 50 amino acids in length; or which encodes a variant of said fragment wherein said variant fragment is at least 95% identical to said fragment, with the proviso that such variant has at most 90%, more preferably 85%, identity with the corresponding mouse or Drosophila sequences when the most similar regions are aligned.

It is another specific object of the invention to provide an isolated polypeptide that comprises the amino acid sequence contained in SEQ ID NO:7, or a polypeptide that possesses an amino acid sequence that is at least 90% identical to the amino acid sequence contained in SEQ ID NO:7, or a fragment thereof that comprises at least 50 amino acids in length, or a variant of said fragment wherein at least 95% of its amino acid residues are identical to said fragment, with the proviso that such variant has at most 90%, more preferably 85%, identity with the corresponding mouse or Drosophila sequences when the most similar regions are aligned.

It is still another specific object of the invention to provide a monoclonal or polyclonal antibody that specifically binds the amino acid sequence contained in SEQ ID NO:7, or a monoclonal or polyclonal antibody which specifically binds to a polypeptide that possesses an amino acid sequence that is at least 90% identical to said amino acid sequence, or a monoclonal or polyclonal antibody which specifically binds a fragment thereof that presents at least one epitope of the amino acid sequence having SEQ ID NO:7, with the proviso that such antibody does not appreciably bind mouse or Drosophila Nkd protein.

### BRIEF DESCRIPTION OF FIGURES

FIG. 1 contains an alignment of the nucleic acid sequence of the coding and 5′ and 3′ non-coding regions of mouse Nkd and human Nkd. [respectively SEQ ID NO:1 and SEQ ID NO:2]

FIG. 2 contains an alignment of the amino acid sequence of human Nkd (“mdap.pep”) and mouse Nkd. [respectively SEQ ID NO:3 and SEQ ID NO:4]

FIG. 3 contains the nucleotide sequence of human Nkd. [SEQ ID NO:5]

FIG. 4 contains the nucleotide sequence of mouse Nkd [SEQ ID NO:6]

FIG. 5 contains the amino acid sequence of human Nkd. [SEQ ID NO:7]

FIG. 6 contains the amino acid sequence of mouse Nkd. [SEQ ID NO:8]

FIG. 7 contains the nucleic acid sequences of the putative promoter of hNkd, as well as the exons for the 5′ UTR, the coding sequence, and the 3′ UTR (exons 1-11). [SEQ ID NO:9 through 22]

FIG. 8 contains a schematic diagram which maps the coding regions and 5′ and 3′ untranslated regions (comprised in exons 1 through 11) of the hNkd gene to an 86 Kb region of human chromosome 16.

FIG. 9 depicts the Wnt/B catenin pathway and identifies particularly genes which promote cell proliferation.

FIG. 10 contains that results of in vitro experiments comparing mNkd mRNA levels in cell cultures exposed to increased amounts of β-catenin over time.

FIG. 11 contains the results of an experiment wherein SW620 cells were treated with β-catenin antisense or reverse control oligos, and ratios of SW620 hNkd, SW620 β-catenin mRNA and control SW620GAPDH mRNA levels measured after treatment.

FIG. 12 shows ratios of hNkd levels in cancer/normal colon tissues.

FIG. 13 depicts schematically a construct used to identify small molecules that modulate the hNkd promoter.

FIG. 14 contains the average of the data obtained in two luciferase reporter assay experiments in 293 cells (wherein “RLV” is a “relative luciferose unit”).

FIG. 15 contains the results of PCR experiments that measured hNkd mRNA levels in a variety of normal tissues wherein the data was normalized to the levels of beta-glucoronidase therein.

FIG. 16 contains the results of real-time PCR experiments that measured hNkd mRNA levels in a variety of human cell lines that included human colon cancer cell lines with results normalized to the level of actin therein.

### DETAILED DESCRIPTION OF THE INVENTION

As discussed supra, the present invention relates to a human or non-human primate homologue of a protein that is involved in Wg/wnt signaling pathway, Nkd, that has been previously identified in Drosophila [Zeng et al., Nature 493 (6771): 789-795 (Feb. 17, 2000)] and in mice [Yan et al., ( 2000)]. More specifically, Zeng et al. (2000)(Id.) reported a Drosophila Nkd gene which encodes a Wg-inducible inhibitor of Wg signaling which antagonizes the Wg/Wnt pathway. Related thereto, Yan et al. (2000)(Id.) reported the identification of a mouse homologue of the Drosophila gene of Zeng et al. (Id.), as well as providing a detailed description of some of its biological properties both in vitro and in vivo and effects on the Wg/Wnt pathway. For example, Yan et al. (2000) discloses that the mRNA levels of the mNkd gene increase in response to Wnt; that mNkd apparently antagonizes the Wnt pathway by blocking the effects of Wnt on beta-catenin and JNK and planar polarity in cell culture as well as in vertebrate Xenopus laevis; and that these effects appear to be mediated by a direct interaction of mNkd with Dishevelled (Dsh), which is a common component of the Wnt and planar polarity pathways.

More specifically, Yan et al. (2000) disclose identification of the mNkd gene by screening a mouse embryonic 9.5 and 10.5 d.p.c. cDNA library using a yeast two-hybrid approach. Particularly, using this screening method, Yan et al. report the identification of several protein fragments which interact with full length Dvl 2 and Dvl 3 proteins. The mNkd protein was identified among these proteins and discovered to contain a single EF-hand calcium binding motif. This motif is disclosed to be most similar to those found in the research family of calcium binding proteins (Zhen et al. (2000). The mNkd protein is disclosed to be 49% similar and 34% identical to the Drosophila Nkd protein reported by Zeng et al. (2000)(Id.), and reportedly contains a similar EF-hand. Yan et al. (2000) also teach or the location of a domain of mNkd which interacts with Dvl in the two-hybrid screen intervenes amino acids 107 to 230, and encompasses the EF-hand motif.

The biological properties of the mouse Nkd reported by Yan et al. (2000)(Id.) are consistent with its role in the Wnt and JNK pathways. For example, as described therein, overexpression of mNkd inhibits Wnt signaling in mammalian cells. In addition, expression of mNkd in mammalian cells activated JNK, a response also seen by expression of Dsh. These results would indicate that mNkd is an activator of the JNK pathway and is involved in Wnt signaling.

As noted above, aberrant Wnt signaling is thought to play a role in some cancers and neurological disorders such as Alzheimer's disease. For example, upregulation of Wnt signaling occurs in some colon cancers. Over-activated Wnt signaling can also be achieved by down-regulating the function of mNkd, which has an inhibitory effect on the Wnt signaling. In some colon cancer cells, mNkd expression may be lower than that in normal cells. Based on this observation, it is anticipated that a human homologue of mNkd will exert similar biological effects, and may be involved in the onset and development of some human cancers or neurological disorders.

Therefore, the identification of a human or non-human primate counterpart of the previously identified mouse and Drosophila Nkd genes would be significant given its significant potential diagnostic and therapeutic applications, especially in view of prior research suggesting that negative regulation of Wnt pathway may be involved in oncogenesis in mammals [Smalley and Date, Canc. Metast. Res. 18(2):215-230 (1999)] and especially cancers such as colon cancer, breast cancer, ovarian cancer among others.

The identification of a human or non-human primate Nkd such a gene or corresponding polypeptide is particularly significant, in the context of potential human therapies, as there would be an expectation that a human Nkd, if one were identified, could regulate the Wnt pathway, in human cells wherein it is implicated in the onset of a number of different cancers. Moreover, the human or primate protein would likely be superior to a non-human protein for human therapy, since it likely would not elicit any immune response in a human subject given its human origin. Therefore, it would potentially be administratable on a chronic basis, e.g. for treatment of cancers involving the Wnt pathway such as breast cancer or neurological disorders involving aberrant Wnt signaling. Also, the human protein would be useful for producing antibodies which would have utility as diagnostic or therapeutic agents, e.g. for detecting levels of expression of hNkd protein as a means of diagnosing cancer or the likelihood of cancer based on aberrant levels of hNkd expression, or for agonizing or antagonizing hNkd expression.

Toward that end, the present invention provides a human or non-human primate Nkd polypeptide and the corresponding nucleic acid sequence, the existence of which was heretofore unknown and could not have been envisioned prior to its invention.

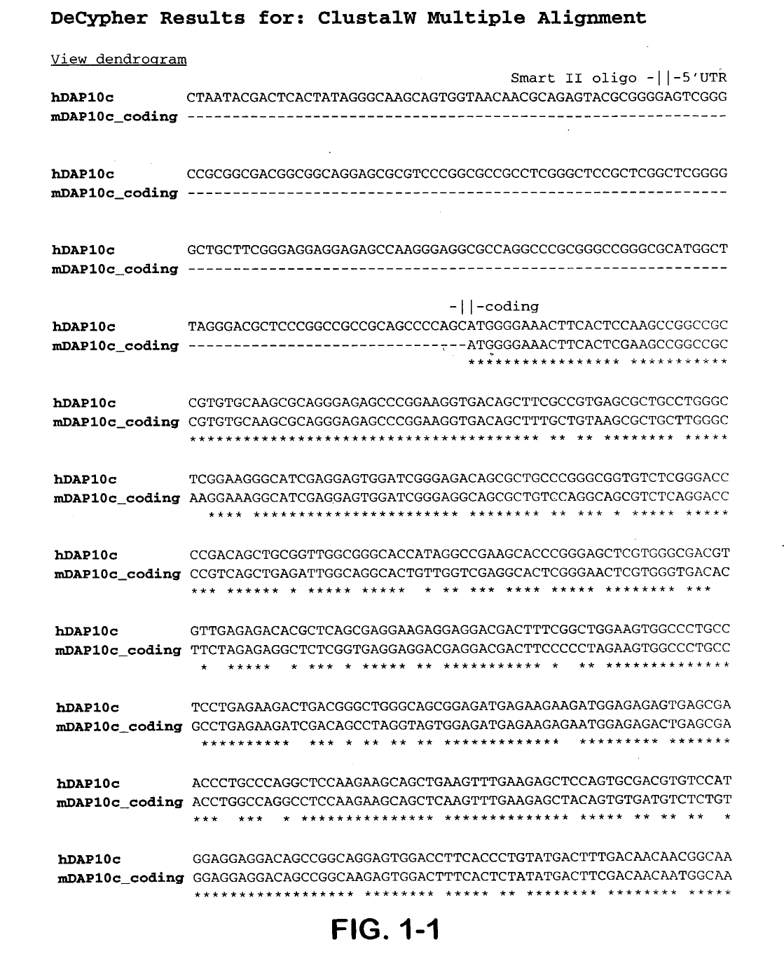

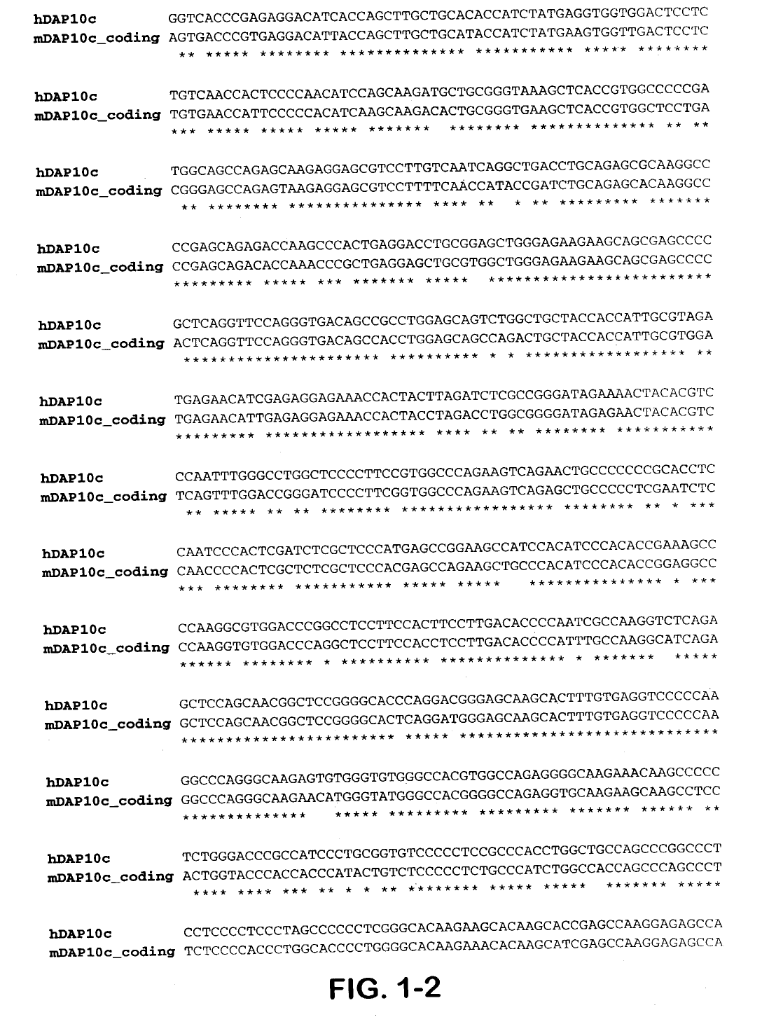

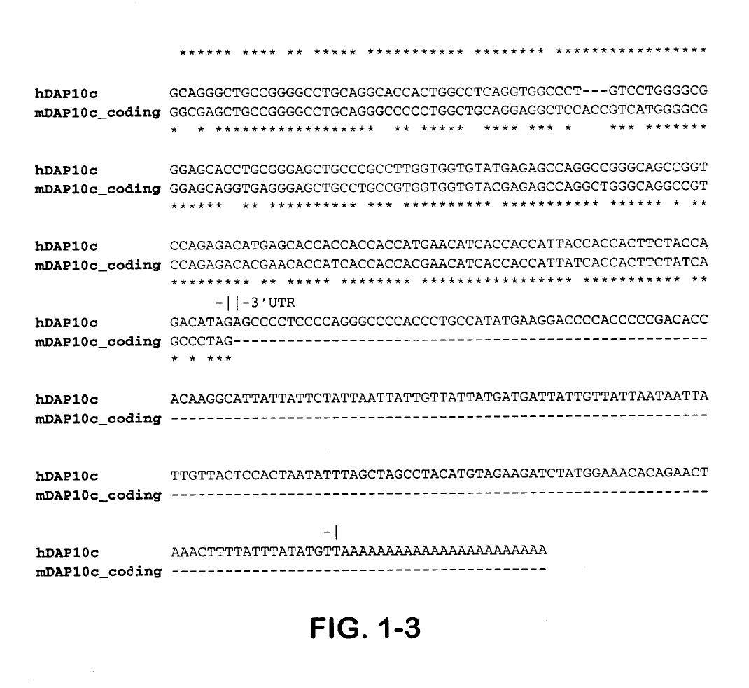

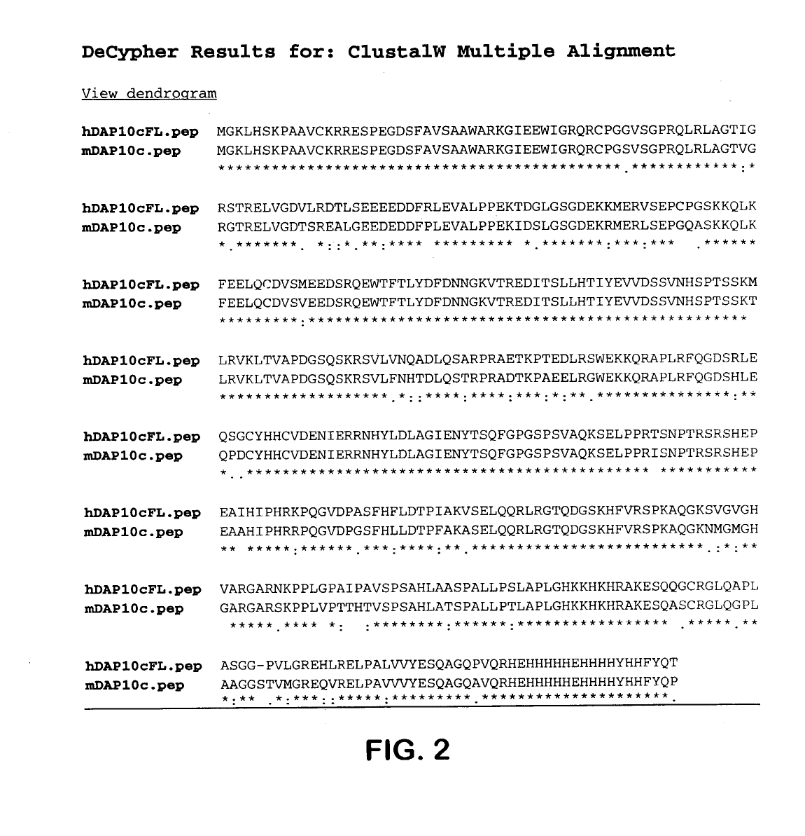

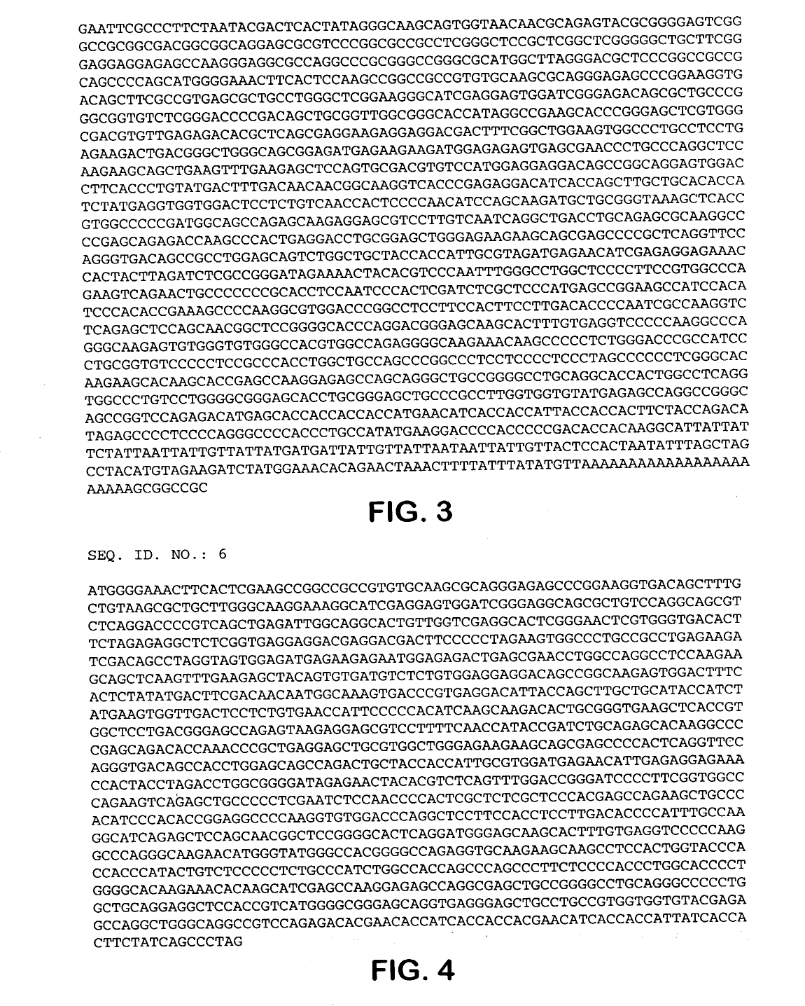

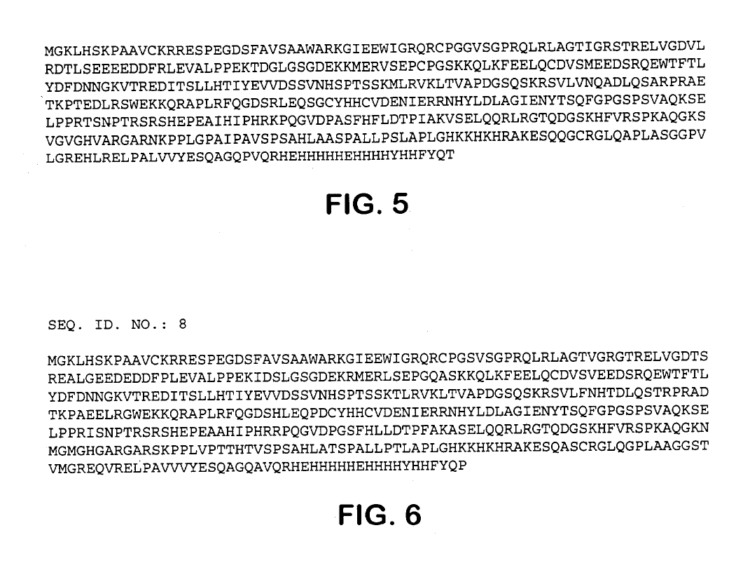

The human Nkd nucleic acid sequence of the protein invention and the corresponding polypeptide was obtained by the methodology disclosed in the examples which follow. Upon sequencing of the entire human Nkd, it was found that the sequence thereof is 85% identical to the mouse Nkd gene, at the nucleotide level, and is 87% identical at the protein level. These proteins contain an identical EF-hand region involved in binding that comprises residues 133 to 168 An alignment of the nucleic acid sequence of the human Nkd gene of the present invention to the nucleic acid sequence of mouse Nkd is contained in FIG. 1. [SEQ ID NO:1 and SEQ ID NO:2 respectively] Additionally, an alignment of the amino acid sequence of the human Nkd protein to the mouse Nkd protein is contained in FIG. 2. [SEQ ID NO:3 and SEQ ID NO:4 respectively]. Additionally the nucleic acid sequences encoding hNkd and mNkd are shown separately in FIGS. 3 and 4 (SEQ ID NO:5 and 6, respectively). Also, the amino acid sequence of hNkd and mNkd are shown separately in FIGS. 5 and 6 (SEQ ID NO:7 and 8, respectively).

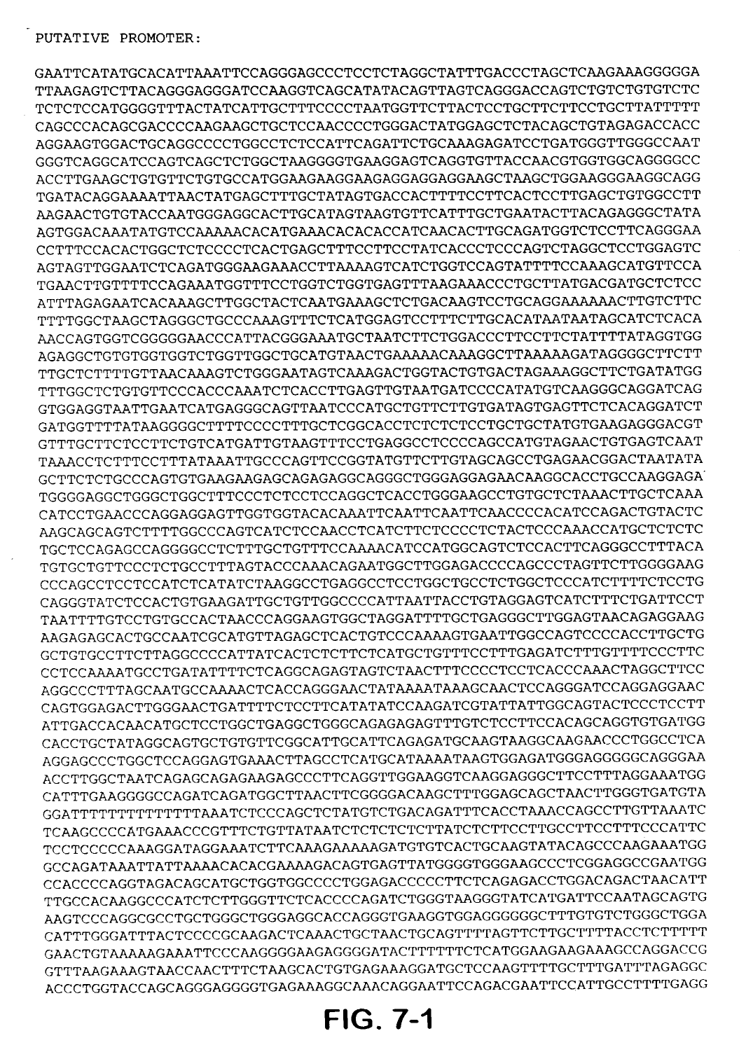

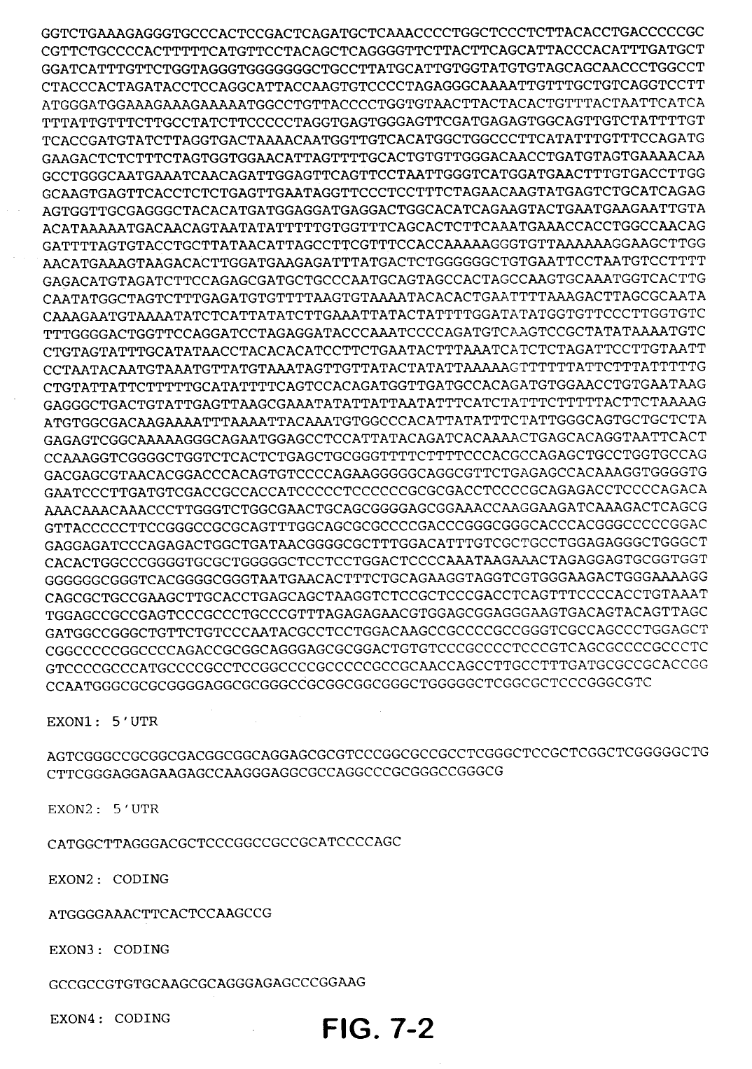

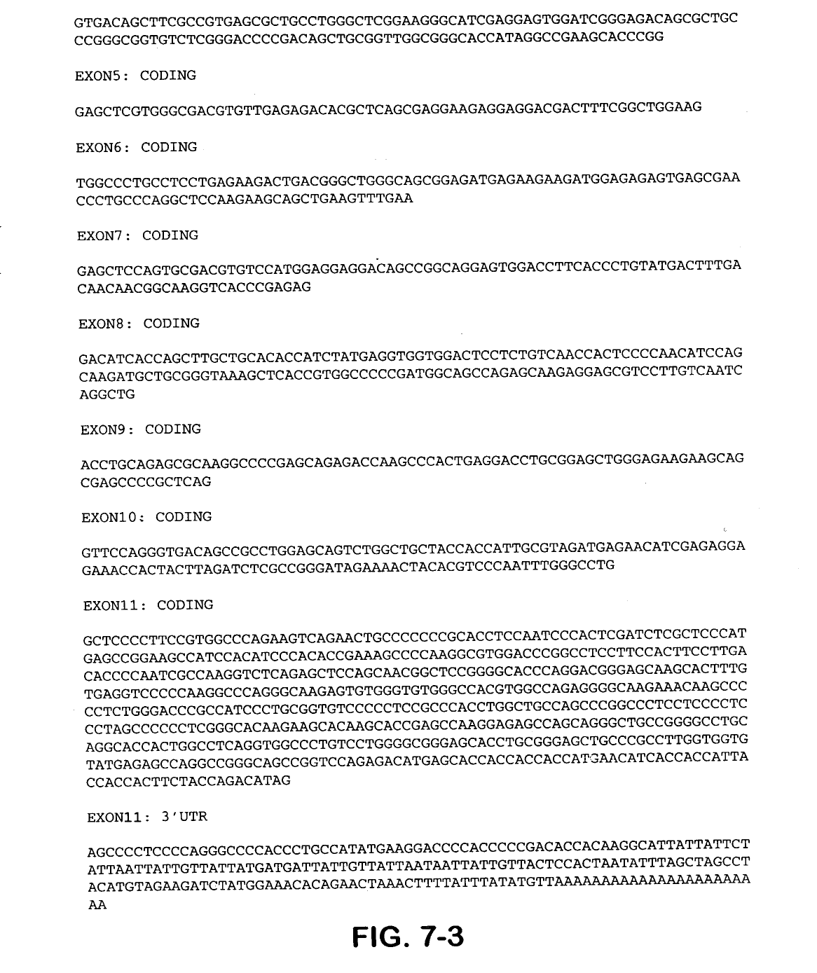

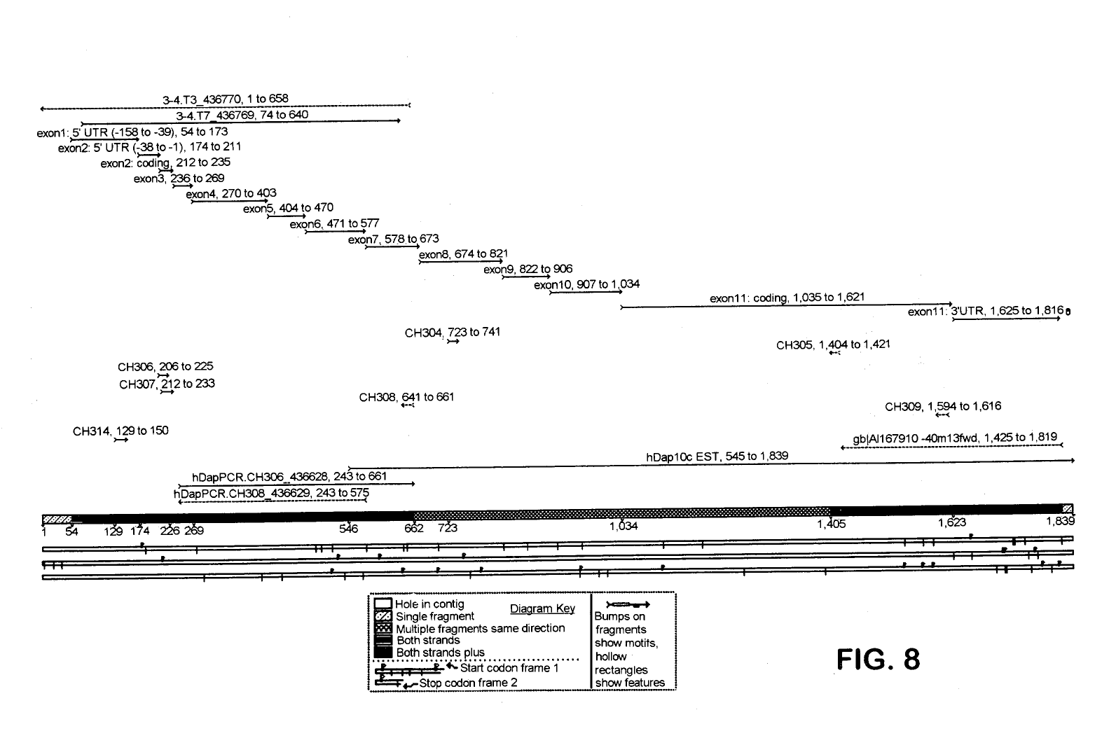

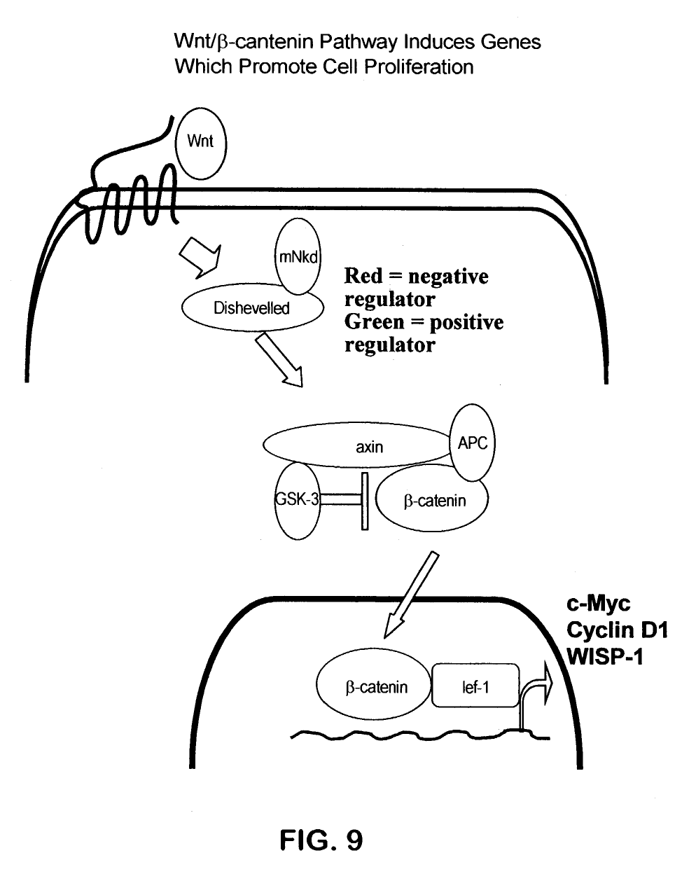

The full length human Nkd nucleic acid sequence of the present invention is comprised on human chromosome 16. Particularly, the entire coding region and the 5′ and 3′ UTRs (comprised in 11 exons) are comprised in an 86 kilobase region found on chromosome 16. A schematic containing the results of this mapping is depicted in FIG. 8. Additionally, the sequences for all 11 exons as well as the putative promoter and 5′ and 3′ UTRs can be found in FIG. 7. [SEQ ID NO:9 through SEQ ID NO:22 respectively]

Given the apparent inducing effect of Wnt ligand on the levels of transcription of mouse Nkd mRNA in cultured mammalian cells, it is hypothesized that the transcription of the human Nkd of the invention may similarly be induced thereby. To further test this hypothesis, the human Nkd promoter sequence was used to blast known databases comprising human genomic sequences. In fact, these genomic searches revealed that this promoter contains several sequence elements that match the inverted consensus for Tcf-binding in other genes (A/T A/T CAAG) [Giese et al., Gevens Dev. 5:2567 (1991)], incorporated by reference in its entirety herein, which region may be necessary for conferring beta-catenin responsiveness. Consistent with this hypothesis is the fact that colon cancer cells that have high levels of beta-catenin also express high levels of human Nkd RNA transcripts. This would suggest that the human Nkd promoter is a direct target of beta-catenin. Related thereto, if it is known that beta-catenin interacts with LEF-1/TCF, APC and conductin/axin and that such interaction may be important in oncogenesis. (Van Kries et al., Nat. Struct. Biol. 7(9): 800-807 (2000); Behrens, J. Ann. NY Acad. Sci. 910:21-33 (2000)). It is also well known that mutations involving beta-catenin are involved in some cancers, especially ovarian, breast and colon cancers. [Wright et al., Int. J. Cancer. 82(5): 62-629 (1999); Li et al., Am. J. Patnol. 153(3): 709-714 (1998); and Fukuchi et al., Cancer Res. 58(16): 3526-3528 (1998)].

Based on this observation, it is anticipated that detection of hNkd, or aberrant expression thereof, wherein aberrant expression includes reduced or increased expression, or the expression of altered forms will provide a means for detecting some cancers, e.g., colon cancer, breast cancer, ovarian cancer, and others involving abnormal Wnt signaling.

Particularly, these results suggest that potential therapies for cancers and other diseases involving aberrant Wnt signaling, among which are colon cancer, ovarian cancer, breast cancer, endometrial cancer, skin cancer, polycystic kidney disease, medulloblastoma pilomatricomas, head and neck cancer, prostate cancer, as well as neurological disorders such as Alzheimer's disease, may include the administration of compounds that modulate hNkd expression, e.g. antibodies, hNkd protein, and fragments or variants thereof; and ribozymes and anti-sense oligonucleotides that specifically target hNkd mRNA transcripts, as well as small molecules that modulate hNkd expression. Preferred examples of cancers that are potentially treatable include colon cancer, breast cancer, and ovarian cancer.

Based on the observed structural similarity between human and mouse Nkd, at both the protein and DNA level, it is anticipated that these proteins and DNAs will exhibit similar biological activities. In particular, it is anticipated that hNkd protein will activate the JNK pathway and inhibit Wnt signaling, similar to mNkd. Based thereon, the invention is directed broadly to novel human and non-human primate Nkd proteins and nucleic acid sequences, variants and fragments thereof, and the use thereof in the design of novel proteins and uses which are discussed in greater detail below. However, prior to specifically describing these proteins, nucleic acid sequences, and uses, the following definitions are provided. Otherwise the terms in this specification have their meanings understood in the relevant field.

“Isolated human or non-human primate Nkd protein” refers to any human or non-human primate Nkd protein that is not in its normal human or primate cellular milieu. This includes by way of example compositions comprising recombinant hNkd, pharmaceutical compositions comprising purified hNkd, diagnostic compositions comprising purified hNkd, and isolated protein compositions comprising hNkd. In preferred embodiments an isolated hNkd protein will comprise a substantially pure protein, in that it is substantially free of other proteins, preferably that is at least 90% pure, that comprises the amino acid sequence contained in SEQ ID NO:3 or natural homologues or mutants having essentially the same sequence. A naturally occurring mutant might be found, for instance, in tumor cells expressing a gene encoding a mutated hNkd protein sequence.

“Native human Nkd protein” refers to a protein that comprises the amino acid sequence contained FIG. 5 and SEQ ID NO:7.

“Native non-human Nkd protein” refers to a protein that is a non-human primate homologue of the protein having the amino acid sequence contained in FIG. 5 and SEQ ID NO:7. Given the phylogenetic closeness of humans to other primates, it is anticipated that human and non-human Nkd proteins will possess amino acid sequences that are highly similar, probably on the order of 95% sequence identity or higher.

“Native mouse Nkd protein” refers to a protein that comprises the amino acid sequence contained in FIG. 6 and in SEQ ID NO:8.

“Isolated human or non-human primate Nkd nucleic acid molecule or sequence” refers to a nucleic acid molecule that encodes human Nkd which is not in its normal human cellular milieu, e.g., is not comprised in the human or non-human primate chromosomal DNA. This includes by way of example vectors that comprise a hNkd nucleic acid molecule, a probe that comprises a hNkd nucleic acid sequence directly or indirectly attached to a detectable moiety, e.g. a fluorescent or radioactive label, or a DNA fusion that comprises a nucleic acid molecule encoding hNkd fused at its 5′ or 3′ end to a different DNA, e.g. a promoter or a DNA encoding a detectable marker or effector moiety. A preferred nucleic acid sequence encodes a human Nkd protein and has the nucleic acid sequence in FIG. 3 and contained in SEQ ID NO:5. Also included are natural homologues or mutants having substantially the same sequence. Naturally occurring homologies that are degenerate would encode the same protein as does SEQ ID NO:5, but would include nucleotide differences that do not change the corresponding amino acid sequence. Naturally occurring mutants might be found in tumor cells, wherein such nucleotide differences result in a mutant Nkd protein. Naturally occurring homologues containing conservative substitutions are also encompassed.

“Variant of human or non-human primate Nkd protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native human or non-human primate Nkd protein wherein sequence identity is as defined infra. Preferably, this variant will possess at least one biological property in common with the native hNkd or primate Nkd protein, e.g. the variant will inhibit Wnt signaling in a mammalian cell, preferably a human or non-human primate cell.

“Variant of human or non-human primate Nkd nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human or non-human primate nucleic acid sequence, wherein “sequence identity” is as defined infra.

“Biologically active fragment of human or non-human primate Nkd” refers to a protein that comprises at least one biological activity of the corresponding native human or non-human primate Nkd, e.g. ability to inhibit Wnt signaling, or ability to present at least one immunogenic epitope of corresponding native human or non-human primate Nkd (as determined by its ability to bind to or elicit anti-hNkd or non-human Nkd antibody); with the further proviso that said biologically active fragment possesses an amino acid sequence, when aligned with the portion of the mouse or Drosophila Nkd protein most similar thereto, that it is not identical therewith, and more preferably possesses at most 90% sequence identity therewith, and more preferably possesses at most 89%, 88%, 87%, 86% or 85% sequence identify therewith, wherein sequence identity is as defined infra.

Biological activity of a positive biologically active hNkd fragment or variant can be determined based on the ability of such fragment or variant to inhibit Wnt signaling, to activate JNK, to interact with disheveled, in vitro or in vivo. Suitable assays are disclosed in the examples infra.

“Fragment of human or non-human primate Nkd nucleic acid molecule or sequence” refers to a nucleic acid sequence corresponding to a portion of the native human Nkd nucleic acid sequence contained in SEQ ID NO:7 or a native non-human primate Nkd nucleic acid molecule, wherein said portion is at least about 50 nucleotides in length, more preferably at least 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, or 1825 nucleotides in length. Preferably such fragments will comprise from about 500 to about 1500 contiguous nucleotide of the human Nkd nucleic acid sequence contained in SEQ ID NO:7, or a non-human primate homologue thereof.

“Antigenic fragments of hNkd or non-human primate Nkd” refer to polypeptides corresponding to a fragment of hNkd or non-human primate Nkd or a variant or homologue thereof that when used itself or attached to an immunogenic carrier that elicits antibodies that specifically bind hNkd or non-human primate Nkd. Typically such antigenic fragments will be at least 20 amino acids in length.

The term “biologically equivalent” is intended to mean that a protein or DNA of the present invention is capable of demonstrating some or all of the same biological properties or similar fashion, not necessarily to the same degree as native human Nkd or non-human primate Nkd protein or DNA. Preferably, biologically equivalent will refer to a variant of hNkd that inhibits Wnt signaling comparably to native human Nkd. As noted, biological activity can be assessed based on the ability of the protein or DNA to other Wnt signaling, activate JNK, to interact with Dsh in vitro or in vivo assays disclosed in the examples infra.

By “substantially homologous” it is meant that the degree of homology of human or non-human primate to another species, preferably another primate, Nkd is greater than that between said hNkd or non-human primate Nkd and any previously reported Nkd (e.g., mouse or Drosophila Nkd).

Sequence identity or percent identity is intended to mean the percentage of the same residues shared between two sequences, referenced to human Nkd when determining percent identity with a non-human Nkd, e.g. mouse Nkd or non-human primate Nkd, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoffet al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human Nkd when determining percent conservation with non-human Nkd, e.g. mNkd, when determining percent conservation. Conservative amino acid changes satisfying this requirement are: R-K; E-D, Y-F, L-M; V-I, Q-H.

Polypeptide Fragments

The invention provides polypeptide fragments of the disclosed proteins. Polypeptide fragments of the invention can comprise at least 8, more preferably at least 25, still more preferably at least 50 amino acid residues of human or non-human primate Nkd, or an analogue thereof. More particularly such fragment will comprise at least 75, 100, 125, 150, 175, 200, 225, 250, 275, 300, 325, 350, 375, 400, 425, 450, 455, 460, 461, 462, 463, 464, 465, 466, or 467 residues of the polypeptide having SEQ ID NO: 7. Even more preferably, the protein fragment will comprise the majority of the native Nkd, i.e. at least about 200 to 400 contiguous residues of hNkd or even more preferably at least 400 to 460 contiguous residues. Preferred protein fragments include those comprising the EF hand. Preferably, such fragment will possess at least one biological activity of human or non-human Nkd protein. In order to establish such biological activity, an in vitro or in vivo assay can be conducted, e.g. to determine whether the fragment binds Dvl, inhibits Wnt signaling by mammalian cells, induces Wnt induced-secondary axes formation in Xenopus laevis embryos, or affects the JNK planar polarity pathway. Exemplary methods are disclosed in the examples of this application.

Biologically Active Variants

Variants of the proteins and polypeptides disclosed herein can also occur. Variants can be naturally or non-naturally occurring. Naturally occurring variants are found in humans or non-human primates species and comprise amino acid sequences which are substantially identical to the amino acid sequence shown in SEQ ID NO:7. Non-human primate homologues or variants of the human Nkd protein can be obtained using subgenomic polynucleotides of the invention, as described below, to make suitable probes or primers to screening cDNA expression libraries from other human or non-human primate cells. Alternatively, primate homologues can be identified by blasting primate DNA databases with the human Nkd protein or DNA sequence.

Non-naturally occurring variants which retain substantially the same biological activities as naturally occurring protein variants are also included here. Preferably, naturally or non-naturally occurring variants have amino acid sequences which are at least 90%, more preferably at least 95%, identical to the amino acid sequence shown in SEQ ID NO:7. More preferably, the molecules are at least 96%, 97%, 98% or 99% identical. Percent identity is determined by a method known in the art. A non-limiting example is the Smith-Waterman homology search algorithm using an affine gap search with a gap open penalty of 12 and a gap extension penalty of 2 BLOSUM matrix of 62. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of hNkd protein or polypeptide variants are of the same type as a protein comprising the amino acid sequence encoded by the nucleotide sequence shown in SEQ ID NO:7, although the properties and functions of variants can differ in degree.

Human or non-human primate Nkd protein variants include glycosylated forms, aggregative conjugates with other molecules, and covalent conjugates with unrelated chemical moieties. Also, hNkd protein variants also include allelic variants, species variants, and muteins. Truncations or deletions of regions which do not affect the differential expression of the hNkd protein gene are also variants. Covalent variants can be prepared by linking functionalities to groups which are found in the amino acid chain or at the N- or C-terminal residue, as is known in the art.

It will be recognized in the art that some amino acid sequence of the hNkd proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the hNkd or non-human primate Nkd polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion Proteins

Fusion proteins comprising proteins or polypeptide fragments of hNkd or non-human primate Nkd can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of hNkd or non-human primate Nkd or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in SEQ ID NO:7 or can be prepared from biologically active variants or fragment of SEQ ID NO:7, such as those described above. The first protein segment can consist of a full-length hNkd or a variant or fragment thereof.

As noted, these fragments may range in size from about 8 amino acids to about 25, 50, 75, 100, 125, 150, 175, 200, 225, 250, 275, 300, 325, 350, 375, 400, 425, 450, 455, 460, 465 amino acids in length.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding an amino acid sequence contained in SEQ ID NO:7 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a coding sequence of the nucleotide sequence shown in SEQ ID NO:5 can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

Human or non-human primate Nkd protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of SEQ ID NO: 1 with the proviso that said sequence is no more than 90% identical with the mouse or Drosophila Nkd sequences, more preferably not no more than about 89, 88, 87, or 86% identical therewith, and most preferably no more than about 85% identical therewith. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in SEQ ID NO:5 for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising human or non-human primate Nkd gene promoter and UTR sequences (SEQ ID NO:9) (SEQ ID NO:10, 11 or 22), operably linked to either Nkd coding sequences or other sequences encoding a detectable or selectable marker. Such promoter and/or UTR-based constructs are useful for studying the transcriptional and translational regulation of Nkd expression, and for identifying activating and/or inhibitory regulatory proteins. The hNkd promoter is apparently regulated by beta-catenin and therefore can be used to render the expression of non-hNkd genes beta-catenin responsive when operably linked thereto.

Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol 153: 163 (1983); Kurtz et al., Mol. Cell. Biol. 6: 142 (1986); Kunze et al., J Basic Microbiol. 25: 141 (1985); Gleeson et al., J. Gen. Microbiol. 132: 3459 (1986), Roggenkamp et al., Mol. Gen. Genet. 202: 302 (1986)); Das et al., J Bacteriol. 158: 1165 (1984); De Louvencourt et al., J Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. 4,837,148; U.S. Pat. No. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761(1985); Gormanetal., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. Pat. No. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12,15, 20, or 50 contiguous nucleotides of the nucleotide sequence shown in SEQ ID NO:5 with the proviso that said sequence is no more than 90% identical with the mouse or Drosophila Nkd sequences, more preferably no more than about 89, 88, 87 or 86% identical therewith most preferably no more than 85% identical therewith. The transcription unit is located upstream to a coding sequence of the endogenous gene. The exogenous regulatory sequence directs transcription of the coding sequence of the endogenous gene.

Human or non-human primate Nkd can also include hybrid and modified forms thereof including fusion proteins, fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains the biological activity of the human or primate Nkd protein. By retaining the biological activity of human or non-human primate Nkd, it is meant that the JNK pathway is activated and/or Wnt signaling is inhibited, although not necessarily at the same level of potency as that of the hNkd isolated as described herein or that of the recombinantly produced hNkd.

Also included within the meaning of substantially homologous is any human or non-human primate protein Nkd which may be isolated by virtue of cross-reactivity with antibodies to the hNkd described herein or whose encoding nucleotide sequences including genomic DNA, mRNA or cDNA may be isolated through hybridization with the complementary sequence of genomic or subgenomic nucleotide sequences or cDNA of the hNkd herein or fragments thereof. It will also be appreciated by one skilled in the art that degenerate DNA sequences can encode human or non-human primate Nkd and these are also intended to be included within the present invention as are allelic variants of hNkd or non-human primate Nkd.

Preferred hNkd of the present invention has been identified and isolated in purified form as described. Also preferred is hNkd prepared by recombinant DNA technology. By “pure form” or “purified form” or “substantially purified form” it is meant that a hNkd composition is substantially free of other proteins which are not hNkd.

The present invention also includes therapeutic or pharmaceutical compositions comprising hNkd or non-human primate Nkd in an effective amount for treating patients with disease, and a method comprising administering a therapeutically effective amount of hNkd. These compositions and methods are useful for treating a number of diseases including cancer. One skilled in the art can readily use a variety of assays known in the art to determine whether hNkd would be useful in promoting survival or functioning in a particular cell type.

In certain circumstances, it may be desirable to modulate or decrease the amount of hNkd expressed. Thus, in another aspect of the present invention, hNkd anti-sense oligonucleotides can be made and a method utilized for diminishing the level of expression of hNkd by a cell comprising administering one or more hNkd anti-sense oligonucleotides. By mNkd anti-sense oligonucleotides reference is made to oligonucleotides that have a nucleotide sequence that interacts through base pairing with a specific complementary nucleic acid sequence involved in the expression of hNkd such that the expression of hNkd is reduced. Preferably, the specific nucleic acid sequence involved in the expression of hNkd is a genomic DNA molecule or mRNA molecule that encodes hNkd. This genomic DNA molecule can comprise regulatory regions of the hNkd gene, or the coding sequence for mature hNkd protein.

The term complementary to a nucleotide sequence in the context of hNkd antisense oligonucleotides and methods therefor means sufficiently complementary to such a sequence as to allow hybridization to that sequence in a cell, i.e., under physiological conditions. The hNkd antisense oligonucleotides preferably comprise a sequence containing from about 8 to about 100 nucleotides and more preferably the antisense oligonucleotides comprise from about 15 to about 30 nucleotides. The hNkd antisense oligonucleotides can also contain a variety of modifications that confer resistance to nucleolytic degradation such as, for example, modified internucleoside linages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, hNkd or non-human primate Nkd can also be linked or conjugated with agents that provide desirable pharmaceutical or pharmacodynamic properties. For example, hNkd or primate Nkd can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject human or primate Nkd protein can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruham, Am. J. Hosp. Pharm. 51:210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern PA, (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject human or primate Nkd protein, fragment or variant thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject hNkd, primate Nkd or variant or fragment thereof are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, hNkd may be therapeutically administered by implanting into patients vectors or cells capable of producing a biologically-active form of hNkd or a precursor of hNkd, Le., a molecule that can be readily converted to a biological-active form of hNkd by the body. In one approach cells that secrete hNkd may be encapsulated into semipermeable membranes for implantation into a patient. The cells can be cells that normally express hNkd or a precursor thereof or the cells can be transformed to express hNkd or a precursor thereof. It is preferred that the cell be of human origin and that the hNkd be human hNkd when the patient is human. However, it is anticipated that non-human primate Nkds may be effective.

In a number of circumstances it would be desirable to determine the levels of Nkd protein or mRNA in a patient. The identification of human Nkd herein along with the previous evidence expression that suggests that Nkd's may be expressed at different levels during some diseases, e.g., cancers, provides the basis for the conclusion that the presence of hNkd serves a normal physiological function related to cell growth and survival. Endogenously produced human Nkd may also play a role in certain disease conditions.

The term “detection” as used herein in the context of detecting the presence of hNkd in a patient is intended to include the determining of the amount of hNkd or the ability to express an amount of hNkd in a patient, the estimation of prognosis in terms of probable outcome of a disease and prospect for recovery, the monitoring of the hNkd levels over a period of time as a measure of status of the condition, and the monitoring of hNkd levels for determining a preferred therapeutic regimen for the patient.

To detect the presence of hNkd in a patient, a sample is obtained from the patient. The sample can be a tissue biopsy sample or a sample of blood, plasma, serum, CSF or the like. It has been found that hNkd is expressed at high levels in some cancers, e.g., colon, breast and lung cancer. Samples for detecting hNkd can be taken from these tissue. When assessing peripheral levels of hNkd, it is preferred that the sample be a sample of blood, plasma or serum. When assessing the levels of hNkd in the central nervous system a preferred sample is a sample obtained from cerebrospinal fluid or neural tissue.

In some instances it is desirable to determine whether the hNkd gene is intact in the patient or in a tissue or cell line within the patient. By an intact hNkd gene it is meant that there are no alterations in the gene such as point mutations, deletions, insertions, chromosomal breakage, chromosomal rearrangements and the like wherein such alteration might alter production of hNkd or alter its biological activity, stability or the like to lead to disease processes. Thus, in one embodiment of the present invention a method is provided for detecting and characterizing any alterations in the hNkd. The method comprises providing an oligonucleotide that contains the hNkd cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the hNkd gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, Taql and Alul. Using the Southern blot protocol, which is well known in the art, this assay determines whether a patient or a particular tissue in a patient has an intact hNkd gene or an hNkd gene abnormality.

Hybridization to a hNkd gene would involve denaturing the chromosomal DNA to obtain a single-stranded DNA; contacting the single-stranded DNA with a gene probe associated with the hNkd gene sequence; and identifying the hybridized DNA-probe to detect chromosomal DNA containing at least a portion of a human hNkd gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The hNkd gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

hNkd gene abnormalities can also be detected by using the PCR method and primers that flank or lie within the hNkd gene. The PCR method is well known in the art. Briefly, this method is performed using two oligonucleotide primers which are capable of hybridizing to the nucleic acid sequences flanking a target sequence that lies within a hNkd gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising hNkd or a fragment thereof is then directly sequenced and analyzed by comparison of the sequence with the sequences disclosed herein to identify alterations which might change activity or expression levels or the like.

In another embodiment, a method for detecting hNkd protein is provided based upon an analysis of tissue expressing the hNkd gene. Certain tissues such as breast, lung, colon and others have been found to express the hNkd gene. The method comprises hybridizing a polynucleotide to mRNA from a sample of tissue that normally expresses the hNkd gene. The sample is obtained from a patient suspected of having an abnormality in the hNkd gene.

To detect the presence of mRNA encoding hNkd protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding hNkd protein or a derivative of the cDNA as a probe, high stringency conditions can be used in order to prevent false positives, that is the hybridization and apparent detection of hNkd nucleotide sequences when in fact an intact and functioning hNkd gene is not present. When using sequences derived from the hNkd cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the hNkd protein, the technique of reverse transcription/polymerization chain reaction (RT/PCR) can be used to amplify cDNA transcribed from mRNA encoding the hNkd protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and hNkd specific primers. [Belyavsky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the hNkd protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the hNkd protein and competitively displacing a labeled hNkd protein or derivative thereof.

As used herein, a derivative of the hNkd protein is intended to include a polypeptide in which certain amino acids have been deleted or replaced or changed to modified or unusual amino acids wherein the derivative is biologically equivalent to hNkd and wherein the polypeptide derivative cross-reacts with antibodies raised against the hNkd protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in in radioinununoassay (RIA), enzyme immunoassays, e.g., enzyme-linked immunosorbent assay (ELISA), fluorescent immunoassays and the like.

Polyclonal or monoclonal antibodies to the subject non-human primate or human Nkd protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, typically a rabbit, hamster or a mouse.

Oligopeptides can be selected as candidates for the production of an antibody to the hNkd protein based upon the oligopeptides lying in hydrophilic regions, which are thus likely to be exposed in the mature protein. Peptide sequence used to generate antibodies against hNkd include:

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for hNkd and do not appreciably bind mouse or Drosophila Nkd protein. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci, US.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3): 169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to hNkd or primate Nkd can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, hNkd or primate Nkd polypeptides of the invention and variants thereof are used to immunize a transgenic animal as described above. Monoclonal antibodies are made using methods known in the art, and the specificity of the antibodies is tested using isolated hNkd or primate Nkd polypeptides.

Methods for preparation of the human or primate Nkd protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system [E. I. du Pont de Nemours Company, Wilmington, Del.) (Caprino and Han, J. Org. Chem. 37:3404 (1972) which is incorporated by reference].

Polyclonal antibodies can be prepared by immunizing rabbits or other animals by injecting antigen followed by subsequent boosts at appropriate intervals. The animals are bled and sera assayed against purified hNkd or primate Nkd protein usually by ELISA or by bioassay based upon the ability to block the action of hNkd or primate Nkd. In a non-limiting example, an antibody to hNkd can block the binding of hNkd to Dishevelled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the hNkd protein by treatment of a patient with specific antibodies to the hNkd protein.

Specific antibodies, either polyclonal or monoclonal, to the hNkd or primate Nkd protein can be produced by any suitable method known in the art as discussed above. For example, murine or human monoclonal antibodies can be produced by hybridoma technology or, alternatively, the hNkd or primate Nkd protein, or an immunologically active fragment thereof, or an anti-idiotypic antibody, or fragment thereof can be administered to an animal to elicit the production of antibodies capable of recognizing and binding to the hNkd or primate Nkd protein. Such antibodies can be from any class of antibodies including, but not limited to IgG, IgA, 1 gM, IgD, and IgE or in the case of avian species, IgY and from any subclass of antibodies.

The availability of isolated human or primate Nkd protein allows for the identification of small molecules and low molecular weight compounds that inhibit the binding of hNkd or primate Nkd to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbence, fluorescence or luminescence as read-outs. [Gonzalez, J. E. et al., Curr. Opin. Biotech. 9:624-631 (1998)].

Model systems are available that can be adapted for use in high throughput screening for compounds that inhibit the interaction of hNkd or primate Nkd with its ligand, for example by competing with hNkd or primate Nkd for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

The therapeutic hNkd or non-human primate Nkd polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90107936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 9 1/02805.

Packaging cell lines suitable for use with the above-described retroviral vector constructs may be readily prepared (see PCT publications WO 95/3 0763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63: 3822-3828 (1989); Mendelson et al., Virol. 166: 154-165 (1988); and Flotte et al., P.N.A.S. 90: 10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleret al., P.N.A.S. 90:11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3: 147-154 (1992); ligand-linked DNA, for example see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 9 1/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

While the invention has been described supra, including preferred embodiments, the following examples are provided to further illustrate the invention.

### EXAMPLE 1

### Identification of Partial Human Nkd Sequence from a Human EST Database

A mouse Nkd nucleic acid sequence having the sequence contained in FIG. 1 (SEQ ID NO: 2), was used to identify a potential human homologue in a human EST database using the blast series of programs [Altschul, S. R. and Lipman, D. J., Proc. Natl. Acad. Sci., USA 87: 5509-5513 (1990)]. Multiple alignment of candidate sequences were conducted using Clustal W alignment [Thompson, J. D. et al., Nucl. Acid. Res. 22: 4673-4680 (1999)].

This search resulted in two hits, Al167910 and H55148. The Al167910 EST was ordered and sequenced. Analysis of the sequence revealed a sequence having substantial similarity to the mouse Nkd sequence, but lacking the 5′ end of the gene.

### EXAMPLE 2

### Cloning Full-Length hNkd

Full-length hNkd was cloned by a combination of laser capture microdissection (LCM) and RT-PCR RACE methodologies. Total RNA was extracted from primary human colon cancer cells by laser capture microdissection (LCM), Arcturus Engineering Inc., Mountain View (Calif.). LCM was carried out by methods well known in the art (See, Simon et al., Am. J. Protol. 156(a): 445-452 (2000)), and provides for the isolation of specific cell types to obtain a substantially homogenous cell sample.

The entire gene including the 5′ and was cloned by the 5′ RACE protocol using as the cDNA template for RACE cDNA synthesized from the total RNA obtained by LCM from a colon cancer patient as described above (RNA prep ID# 100/sample 1b3521 sample Name UC-C2CA) according to the manufacturers protocol (Clontech SMART RACE cDNA Amplification Kit, K1811-1) which is described below:

5′ RACE Protocol:

cDNA template for RACE was synthesized from RNA isolated by LCM from a cancer patient (RNA prep ID#100/Sample ID352/Sample name UC-C2CA) according to manufacturer's protocol (Clontech SMART RACE cDNA Amplification Kit, K1811-1). Amplification was performed with Clontech Advantage GC-cDNA PCR kit (K1907-1) at 1M GC according to manufacturer's protocol, using cDNA template above, universal primer mix provided by manufacturer and a primer specific for human Nkd (CH308: CTTGCCGTTGTTGTCAAAGTC). PCR was carried out for 30 cycles of 94° C., 0.5 min/58° C., 0.5 min/68° C.. 2 min, followed by 10 cycles of 94° C., 1 min/58° C., 1 min/68° C. 2 min. A final round of extension was carried out at 72° C. for 10 min. The PCR products were cloned into pCR-TOPO4 (Invitrogen) and transformed into E. coli. Bacterial colonies harboring the correct 5′RACE product were identified by PCR screening using nested primers (CH306: CCCAGCATGGGGAAACTTCA and CH308: CTTGCCGTTGTTGTCAAAGTC).

This result in isolation of the full length hNkd gene including 5′ and 3′ untranslated regions. This sequence is contained in FIG. 1 and 3, and SEQ ID NO:1, and SEQ ID NO:5 respectively.

The nucleic acid sequence of human Nkd (including 5′ and 3′ untranslated regions) are aligned by Clustal W Multiple Alignment in FIG. 1. It can be seen from this alignment that hNkd is 85% identical to mNkd at the nucleotide level. Additionally, the hNkd and mNkd DNA sequences are shown separately in FIGS. 3 and 4 (SEQ ID NO:5 and 6).

Upon review of the nucleotide sequence, the protein sequence was discerned. This sequence is shown in FIG. 5, and SEQ ID NO:7. It can be seen therefrom that the hNkd protein is 470 amino acids in length.

The amino acid sequence of hNkd and mNkd are aligned in FIG. 2. It can be seen from this alignment that the amino acid sequence of mouse and human Nkd are 87% identical at the protein level, and that mouse Nkd contains one less amino acid residue than the human Nkd protein. Additionally, the amino acid sequence for hNkd and mNkd are depicted separately in FIGS. 5 and 6 (SEQ ID NO:7 and 8). It can also be seen that these proteins contain an identical EF-hand region corresponding to residues 133 to 168 involved in binding.

### EXAMPLE 3

### Mapping of hNkd to a Human Chromosomal Region

The full-length Nkd nucleic acid sequence obtained in the previous example was used to blast a Genbank genomic sequence database to identify the chromosomal region that comprises hNkd. This sequence resulted in two BAC hits from human chromosome 16. As shown in FIG. 7 and 8, the entire coding region of hNkd consists of 11 exons. These exons can be mapped to an 86 Kb region on human chromosome 16. Sequence analysis revealed that the genomic sequence comprises the identical coding region and 3′ untranslated region as the hNkd sequence cloned by 5′ RACE. The only sequence differences are two nucleotide changes in the 5′ UTR compared to the Genbank genomic sequence. The nucleotide sequence of the 11 exons, including the untranslated and coding regions, are contained in FIG. 7. (SEQID NOs:9-22). It is unknown at this time whether the two nucleotide changes in the 5′ UTR have any effect on hNkd expression.

### EXAMPLE 4

### Preparation of Fusion Proteins

GST fusion proteins are expressed in E. coli strain BL21 DE3 (plyS) and purified with glutathione beads (Pharmacia). Myc-hNkd protein is prepared by in vitro transcription and translation using TNT coupled reticulocyte lysate system (Promega) in the presence of ³⁵S-metheonine. The ³⁵S-labeled hNkd is precipitated for 3 hours at 4° C. by anti-Myc antibody and protein A beads or by GST fusion proteins immobilized on glutathione beads.

### EXAMPLE 5

### Inhibition of Wnt Signaling by hNkd

293 cells were co-transfected with a LEF luciferase reporter expressing firefly luciferase, a LEF-1 expressing vector, a pRL-TK vector (Promega) expressing Renilla luciferase as a transfection control, and the following plasmids: pCDNAHis3C β-galactosidose; pCGWnt-1 plus pCDNAHis3C β-galactosidose; pCGWnt-I plus pC52+MychNkd; pC52+MychNkd alone. The LEF-1 luciferase reporter activities of each sample are determined and normalized using the dual-luciferase reporter assay system according to the manufacturer's instructions (Promega).

Particularly, 293 cells were grown in 12 well plates. Cells were transfected with expression constructs of LEF-1, LEF-1 reporter, Renilla and β-galactosidose, hNkd, Wnt-1 plus β-galactosidose, or Wnt-1 plus hNkd. Thirty-six hours post-transfection relative luciferase activities were measured.

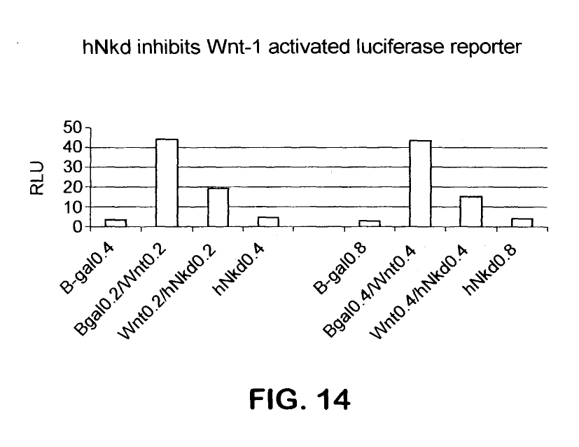

The average of data of the experiments are contained in FIG. 14. Based on these results, it can be seen that hNkd inhibited Wnt-1 signaling in a luciferase reporter assay in 293 cells (wherein RLV in the figure refers to relative luciferase unit).

### EXAMPLE 6

### Expression of hNkd mRNA in Normal and Cancerous Human Cell Lines and Tissues

Normal Tissues

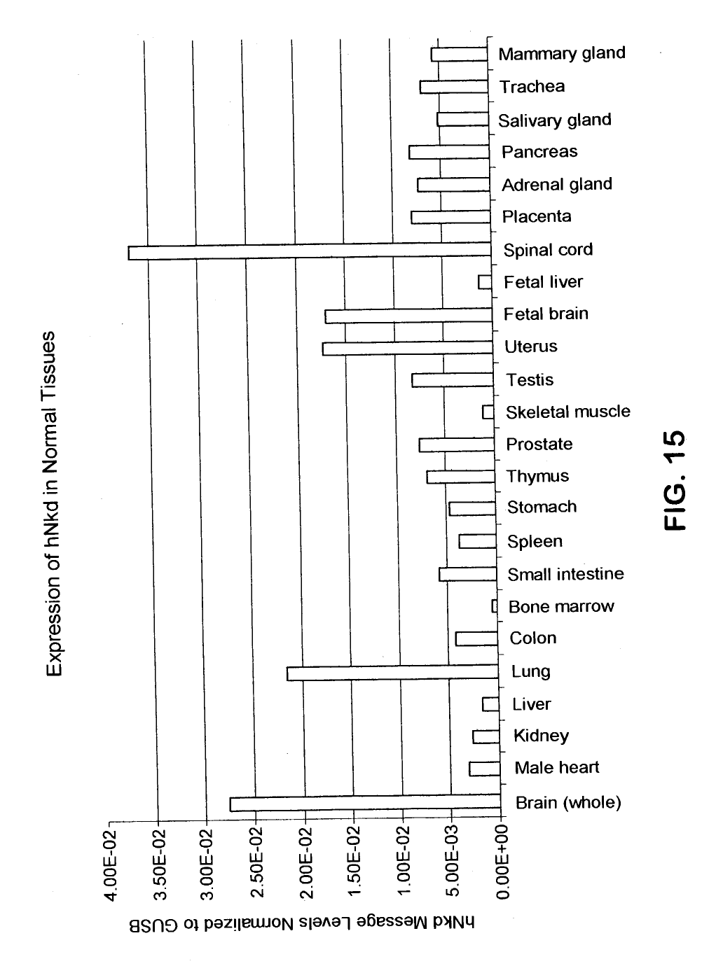

hNkd mRNA levels were measured by real-time quantitative PCR in brain, male heart, kidney, liver, lung, colon, bone marrow, small intestine, spleen, stomach, thymus, prostate, skeletal, muscle, testis, uterus, fetal brain, fetal liver, spinal cord, placenta, adrenal gland, pancreas, salivary gland, trachea and mammary gland. The expression of hNkd in all tissues was normalized to the levels of beta-glucoronidase. The ratios ranged from 1.00E-03 (lowest expression in bone marrow) to 4.00E-02 (highest expression in spinal cord). The results of this experiment are in FIG. 15 and show that hNkd basal mRNA levels are low in normal tissues.

Cancer Cell Lines

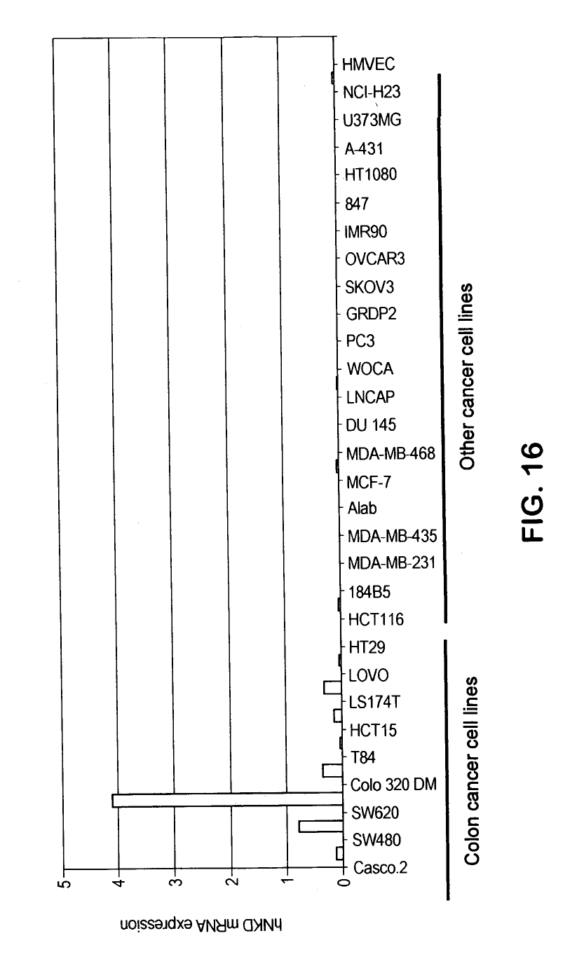

A collection of human cell lines including Caco. 2, SW480, SW620, Colo 320 DM, T84, HCT15, LS174T, LOVO, HT29, HCT116 (colon cancer cell lines); 184B5 (primary breast cell lines); MDA-MB-231, MDA-MB-435, Alab, MCF-7, MDA-MB-468 (breast cancer cell lines); DU145, LNCAP, WOCA, PC3, GRDP2 (prostate cancer cell lines); SKOV3, OVCAR3 (ovarian cancer cell lines); IMR90 (primary fibroblast cell line); 847 (SV40-transformed fibroblast cell line); HT1080 (fibrosarcoma cell line); A-431 (epidermoid carcinoma cell line); U373MG (glioblastoma cell line); NCIH23 (non-small cell lung cancer cell line), HMVEC (endothelial cell line), were grown in media as recommended by the ATCC. After extraction of total RNA, cDNA was synthesized with oligo dT primers and used in quantitative real-time PCR analyses to measure the expression levels of hNkd mRNAs normalized to the levels of actin mRNAs therein. These results are contained in FIG. 16 and show that hNkd mRNA was expressed at high levels only in colon cancer lines and not in other types of human cancer cell lines tested.

### EXAMPLE 7

### Activation by JNK by hNkd

The JNK assay is carried out as described [Boutros et al., Cell 94:108-118 (1998)] with modifications. NIH3T3 cells are grown in six-well plates to exponential growth in DMEM medium with 10% calf serum. The Cells are transfected using LipofectAMINE plus reagent (Lifetech) according to the manufacturer's protocol. Twenty two hours after transfection, cells are lysed in SDS sample buffer. Equal amounts of samples are separated by Tris-Glycine polyacrylamide gel (Novex) and transferred onto nitrocellulose membrane. The membrane is blotted with PhosphoPlus c-Jun (Ser63) II antibody (New England Biolabs) which recognizes the phosphorylated serine at position 63 in the N-terminus of c-Jun. The same membrane is then stripped and blotted with antiXpress antibody (Invitrogen) to detect the amount of X-press tagged hNkd and β-galactosidase expressed. The same membrane is stripped again and blotted with anti-GAP antibody to detect the amount of GAP in each sample as loading control.

It is expected that there will be an increase in intensity of the phosphorylated c-Jun band.

### EXAMPLE 8

### Expression of hNkd in Mammalian Tissues

The expression of hNkd in mammalian tissues is investigated using a multiple tissue Northern Blot (Clontech). The Northern Blot is hybridized with a radioactively labeled fragment of hNkd. The fragment includes the Dishevelled binding domain of hNkd. The tissues that are to be analyzed include, heart, brain, spleen, lung, liver, muscle, kidney and testis.

### EXAMPLE 9

### hNkd Interaction with Dishevelled

Cos7 cells expressing hNkd, hNkd-delta EF hand, or GFP gene in vector pCDNA3.IHisC (In Vitrogen) are lysed in buffer containing 150 mM NaCl, 20 mM Tris HCl pH 7.5, 0.1% Triton with protease inhibitor cocktail tablets (Roche). Total cell lysate are immunoprecipitated with monoclonal Dvl antibodies 1, 2, and 3 (Santa Cruz, Calif.) and blotted with Xpress antibody. Anticipating that the EF domain of hNkd behaves similarly to that of mNkd, then Nkd mutations in the EF-hand should not have a significant effect on the binding of hNkd to Dvl. To test this hypothesis, cell lysate from Cos7 cells expressing Myc tagged Dishevelled an fragments thereof containing the DD2, Nor C terminal domains of Dsh and hNkd mutants are immunoprecipitated with Myc antibody (Roche) and blotted with Xpress antibody hNkd mutants are produced by site-specific mutogenesis having one or two amino acids in the EF-hand changed relative to native hNkd.

In another experiment, Myc-tagged hNkd protein, labeled with ³⁵S, is immunoprecipitated with anti-Myc antibody or precipitated by GST fusion proteins. The immunocomplexes are subjected to electrophoresis and autoradiophy. The GST fusion proteins from bacteria are separated on SDS-PAGE gel and Coomassie blue stained. It is anticipated that hNkd will behave similarly to mNkd, and that ³⁵S-hNkd will associate with fragments of Dsh including the PDZ and adjoining N-terminal basic amino acid residues but not the N or C terminal domains of Dsh.

### EXAMPLE 10

### Effects of hNkd and EF-Hand Mutations of hNkd on the Wnt Responsive LEF-1 Reporter Activities in Mammalian Cells

mNkd transcription is Wnt and lithium chloride treatment inducible. It is anticipated that hNkd transcription will be similarly inducible. To test this hypothesis, HEK 293 cells are seeded at 2×10⁵/well in 12-well culture plates. Each well is transfected using LipofectaminePlus (Life Science) with a total of 0.54 μg of DNA. The transfect DNA includes 0.02 μg of LEF-I, 0.2 μg of luciferase reporter [Hsu et al., Mol. Cell. Biol. 1918(1998)], 0.02 g of pRL-TK (Promega) and a combination of 0.1 μg of pCGWnt-I with either 0.2 μg of pCDNA3.IHisC GFP, hNkd, or its derivatives. In samples comprising mutations in the EF hand or deletion of the EF hand where no Wnt-1 is transfected, GFP, hNkd or derivatives of hNkd alone at 0.3, or 0.1 μg is transfected. LEF-1 luciferase reporter activities are determined and normalized using dual-luciferase reporter assay system (Promega). It is anticipated that the results will show that hNkd does not inhibit beta-catenin activated LEF-1 reporter expression, and that hNkd negatively regulates canonical Wnt signaling and that point mutations as well as deletion of the EF hand impairs the ability of hNkd to inhibit the Wnt mediated transcriptional responses.

Mouse liver epithelial cells are treated with either Wnt-3a conditioned medium or Neo control medium for 9, 19.5 or 27 hours. Growth medium with or without 40 mM LiCl is used to treat cells for 16 hrs. Suitable primers are used in RT-PCR to amplify hNkd and GAPDH respectively.

### EXAMPLE 11

### hNkd Effects in Xenopus Embryo

The developing Xenopus embryo provides an effective in vivo assay for Wnt signaling, as ectopic ventral activation of this pathway induced ectopic dorsal structures. Ventral injection of 5-10 pg of Xwnt-8 mRNA for mNkd has been shown to induce secondary axes in over 60% of embryos. Consistent with the ability of mNkd to inhibit canonical Wnt-induced, LEF-I dependent transcription, injection of 35 pg of hNkd mRNA is expected to suppress the activity of co-injected Xwnt-8. Co-injection of higher doses of hNkd should result in even fewer secondary axes, as it has been reported that the ventral expression of very high doses of Drosophila Nkd induce ectopic head structures [Zeng et al; (2000)].

A vertebrate cognate of the Drosophila planar polarity pathway controls convergent extension movements during vertebrate development. In both Xenopus and Drosophila, hyperactivation of this pathway elicits cell polarity phenotypes that are independent of canonical Wnt signaling. Overexpression of wild-type Dsh of Frizzled (Fz) in Drosophila disrupts epithelial planar polarity, while in Xenopus, overexpression of wild-type Xdsh, Xfz-8, or Xfr-7 disrupts cell polarity and inhibits convergent extension. As such, convergent extension represents an effective in vivo assay of vertebrate planar polarity signaling.

Consistent with its expected ability to activate JNK in vitro, overexpression of hNkd should inhibit the normal elongation of Xenopus embryos in a manner similar to Drosophila and mouse Nkd. To more directly assess the effects of hNkd on convergent extension, open-face Keller explants of the dorsal mesoderm are examined. Xenopus embryos are injected with in vitro transcribed mRNAs into either two dorsal or two ventral blastomeres at the four-cell stage and are reared in 1/3×MMR to stage 30 for scoring of phenotypes. Keller explants are cut at st. 10.25 and cultured under coverglass in 1× Steinberg's until st. 20.

Control Xenopus embryos are injected ventrally with 5-10 pg of Xwnt-8 mRNA developed with secondary axies. Co-expression of hNkd with Xwnt-8 should decrease the frequency of secondary axes formation as well as the ratio of complete secondary axes compared to Xwnt-8 alone.

As dorsal expression of mNkd in developing Xenopus embryos inhibited the normal elongation and straightening of the anteroposterior axis, it is anticipated similar results with hNkd. The normal formation of anterior structures such as in embryos which express mNkd indicates that the phenotype is not the result of ventralization, suggesting that mNkd inhibits convergent extension. Similarly, although control explants of the dorsal marginal zone elongate and change shape significantly, explants expressing mNkd failed to elongate or to change shape. Downstream activation of the canonical Wnt pathway by co-expression of DN-GSK3 did not rescue the effects of mNkd on convergent extension.

The results of this experiment should show that explants made from control embryos elongate significantly, while explants made from embryos expressing hNkd fail to elongate. These anticipated results are similar to those elicited by over-expression of other wild-type components of the planar polarity cascade, including Xdsh, Frizzled-8, Frizzled-7, and Wnt-11.

Because hNkd is expected to be a potent inhibitor of the Wnt pathway, it is important to test whether the effects of hNkd on convergent extension may result from that activity. An experiment is performed in which DN-GSK3, which should strongly activate canonical Wnt signaling (Pierce, 1995), with hNkd, but not provide for rescue of convergent extension found. Combined with the ability of mNkd to activate JNK, these data indicate that mNkd inhibits convergent extension by over-stimulating the planar polarity signaling cascade.

Although the inventors are not bound by a particular mechanism of action, based on experiments conducted with mNkd it is anticipated that canonical Wnt signaling will be inhibited by hNkd herein, that it will activate the non-canonical planar polarity pathway, and that hNkd will bind to Dsh. Therefore, it is hypothesized that hNkd may be involved in shunting Dsh out of the canonical pathway and into the planar pathway. This will make hNkd a critical regulator of the decision fork between canonical and non-canonical Wnt pathways.

### EXAMPLE 12

### Antibodies Capable of Binding to mNkd

Antibodies to hNkd, or a fragments thereof, are prepared as follows. Rabbits or other suitable mammals or animals are injected with hNkd polypeptide or an antigenic fragment comprising at least 20 amino acids of the protein having SEQ. ID. NO.: 7 thereof followed by subsequent boosts at appropriate intervals. The animals are bled and sera is assayed against purified hNkd protein or a fragment thereof. Peptide sequences for producing anti-hNkd antibodies include fragments of hNkd comprising at least 20 contiguous amino acids thereof.

Preferably, antibodies are selected that specifically bind to hNkd but which do not appreciably bind to mouse or Drosophila Nkd. Monoclonal antibodies that specifically bind hNkd but do not bind mouse or Drosophila Nkd are obtained by producing hybridomas from B cells derived from immunized animals, and screening for those that produce monoclonal antibodies that specifically bind human or non-human primate Nkd, but which do not substantially bind mouse or Drosophila Nkd. Antibodies are preferably selected that bind to different portions of hNkd, e.g., the EF hand.

### EXAMPLE 13

### Treatment of Breast Cancer using Anti-hNkd Antibody

A patient having a cancer involving aberrant Wnt signaling, in particular breast cancer is treated by administration of a therapeutically effective amount of an anti-hNkd antibody according to the invention contained in a pharmaceutically acceptable carrier.

This administration is repeated as necessary until the desired therapeutic response is achieved.

### EXAMPLE 14

### Detection of Cancer Involving Wnt Signaling

A subject is screened for the presence of a cancer involving aberrant expansion of hNkd by measuring the amount of hNkd protein in the serum of such subject using an anti-hNkd antibody according to the invention and correlating these levels to a control serum sample (normal patient serum). A positive diagnosis is made if these levels substantially exceed hNkd levels in the normal serum sample.

### EXAMPLE 15

### mNkd mRNA is Induced by Wnt Ligand

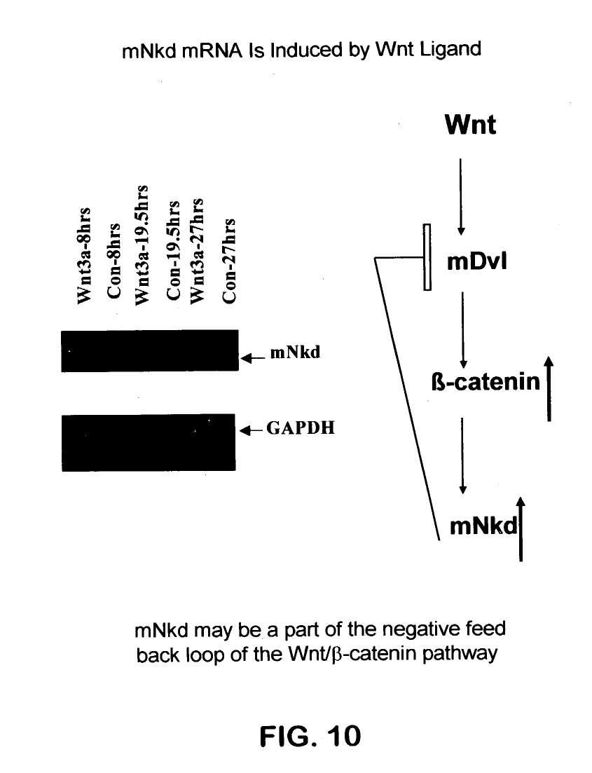

An experiment was conducted which determined the effect on hNkd mRNA levels upon exposure of cell cultures to increased amounts of β-catenin. These results are contained in FIG. 10 and show that increased amounts of β-catenin at time periods of eight hours, 19.5 hours and 27 hours results in the induction of mNkd expression as evidenced by increased mNkd mRNA levels. By control, the control gene (GAPDH mRNA) showed no such increase in mRNA levels. These results suggest that mNkd may be part of a negative feedback loop of the Wnt/β-catenin pathway.

### EXAMPLE 16

### Effect of Antisense Oligos on Ratios of hNkd and B-Catenin mRNA Levels

In order to confirm the involvement of the hNkd gene in certain cancers, particularly colon cancer, a colon cancer cell line, SW620, was treated with β-catenin RC/AS oligos. Specifically, SC620 cells were tested with the following antisense oligos, CHIR30-5AS 5′-ACTCAGCTTGGTTAGTGTGTCAGGC-3′, and the reverse control oligos (CHIR30-5RC 5′-CGGACTGTGTGATTGGTTCGACTCA-3′). This experiment is described as follows.

Antisense Assay: SW620 cells were grown in DMEM supplemented with 10% FBS. Cells were transfected with antisense β-catenin oligo (AS) or reverse control oligo (RC; same nucleotide sequence with different orientation) to a final concentration of 100 uM using the in house proprietary transfection reagents. 48 hrs. after transfection, cells were washed with PBS twice and total RNA was prepared using RNeasy mini kit (Qiagen). Reverse-transcription and quantitative RNA analyses were performed by using a LightCycler system from Roche Molecular Biochemicals.

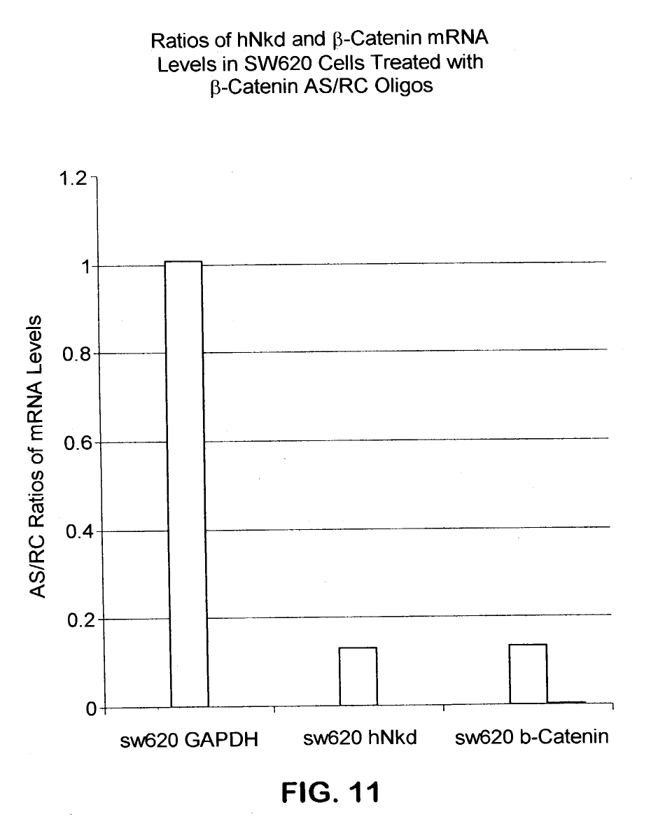

hNkd mRNA Level is Regulated by β-Catenin. SW620 is a colon cancer cell line with up-regulated cytosolic β-catenin level due to mutations in the genes of the Wnt signaling pathway. β-catenin antisense treatment of the SW620 cells specifically down regulates the mRNA level of β-catenin, as demonstrated by the over 80% reduction in the β-catenin mRNA level of cells treated with β-catenin antisense (AS) compared to that of cells treated with reverse control oligo (RC). These results are shown in FIG. 11. β-catenin protein level is also down regulated in the β-catenin antisense treated samples (data not shown). Similarly, the hNkd mRNA level of cells treated with β-catenin antisense is also 80% lower than that of those cells treated with the reverse control oligo. As a negative control, GAPDH mRNA levels were determined and should not be affected by either β-catenin antisense or reverse control treatment. Therefore, the ratio of GAPDH of cells treated with β-catenin antisense versus that of cells treated with reverse control oligo is equal to one.

### EXAMPLE 17

### Distribution of hNkd RNA in Colon Cancer Samples and Normal Tissues

Based on our observation that hNkd mRNA levels are upregulated by increases in β-catenin in cell culture, and further based on our identification of numerous TCF-binding elements in the hNkd promoter, we predicted that hNkd levels would be elevated in colon cancer patients relative to normal persons. In fact, as described in greater detail below, it was found that hNkd RNA levels are elevated significantly in about 70% of colon cancer samples relative to normal cultures.

Quantitative Analysis of hNkd Messenger RNA Levels in Colon Cancer and Patient Matched Normal Samples: mRNA was isolated from laser-capture microdissection (LCM) prepared colon cancer samples. Reverse-transcription reaction was performed using RETROscrip kit (Ambion) with the random primers provided with the kit. Quantitative RNA analyses were performed by using a LightCycler system from Roche Molecular Biochemicals.

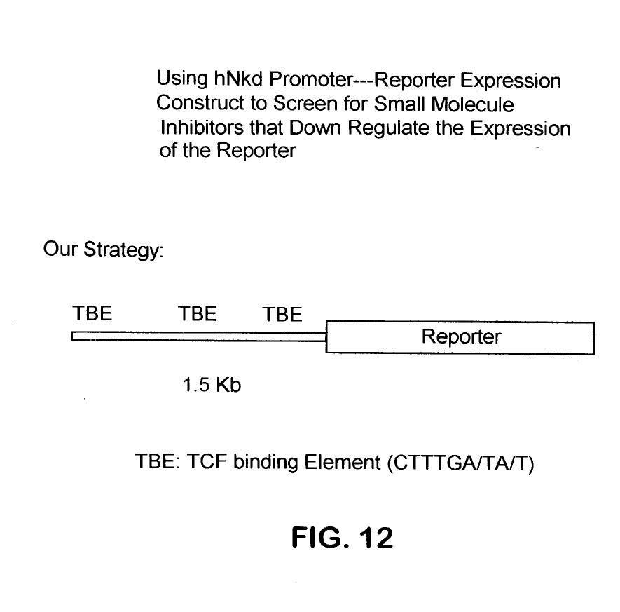

hNkd mRNA Levels Are Up-Regulated in Colon Cancer Patients. Over 80% of colon cancer patients have aberrant regulation of the Wnt pathway that results in the up regulation of cytosolic β-catenin levels. Since hNkd mRNA level is regulated by the amount of β-catenin, we investigated hNkd mRNA levels in 28 pairs of colon cancer and patient matched normal samples. These results are contained in FIG. 12 and indicate that 68% of colon cancer patients have elevated hNkd mRNA levels, consistent with our finding that β-catenin regulates hNkd mRNA level.

### EXAMPLE 18

Based on the foregoing results, nNkd expression is used as a marker to diagnose colon cancer. A colon sample is obtained from a subject to be tested for colon cancer. The levels of hNkd RNA are measured and compared to hNkd RNA levels in a normal control. A positive colon cancer diagnosis is made based on whether the subject exhibits increased mNkd RNA levels relative to the control.

### EXAMPLE 19

### Use of hNkd Promoter to Screen for Small Molecule Inhibitors that Downregulate the Expression of a Reporter

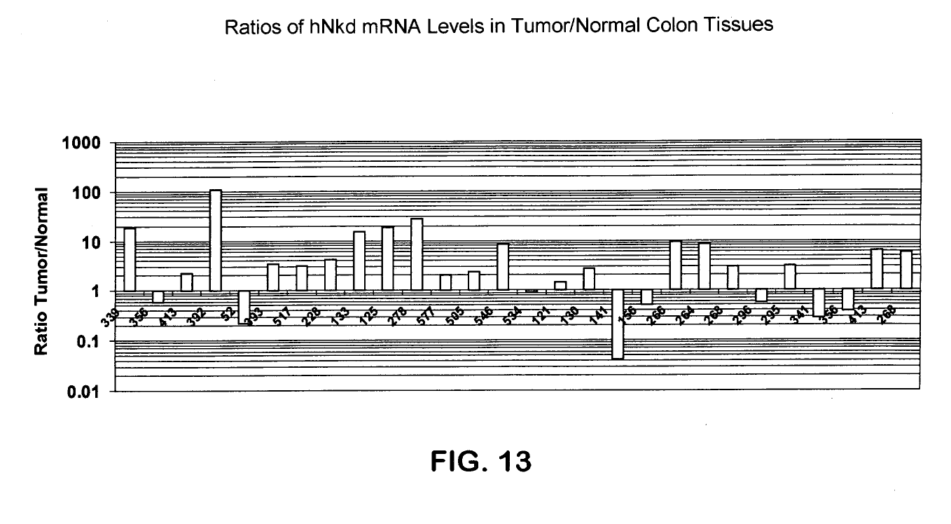

The hNkd promoter is used to identify small molecules that downregulate the expression of a reporter gene operably linked to the promoter. For example, a DNA construct containing the hNkd promoter which has been found to contain multiple TCF binding elements (CTTTGA/TA/T) (shown schematically in FIG. 13) is operably linked to the coding sequence of green fluorescent protein, and is introduced into a suitable mammalian cell line. This cell line is then contacted with different small molecules and the effects on reporter expression measured. Small molecules which downregulate reporter expression are selected. These molecules potentially are useful in treating diseases, especially cancers, involving abnormal regulation of the Wnt/β-catenin pathway such as colon cancer.

The present invention has been described with reference to specific embodiments. However, this application is intended to cover those changes and substitutions, which may be made by those skilled in the art without departing from the spirit and scope of the appended claims.

### SEQUENCE LISTING

SEQUENCE ID NO: 1

DNA sequence of “hDAP10” (hNkd) contained in ClustalW Alignnment of human and mouse Nkd DNA sequences. (hNkd nucleic acid sequence of FIG. 1).

SEQUENCE ID NO: 2

DNA sequence of “mDAP10C” (mNkd) contained in ClustalW Alignment of human and mouse Nkd DNA sequences. (mNkdnucleic acid sequence of FIG. 1).

SEQUENCE ID NO: 3

Amino acid sequence for hNkd in ClustalW alignment of human and mouse Nkd amino acid sequences. (hNkd amino acid sequence of FIG. 2.).

SEQUENCE ID NO: 4

Amino acid sequence for mNkd in ClustalW alignment of human and mouse Nkd amino acid sequences. (mNkd amino acid sequence of FIG. 2).

SEQUENCE ID NO: 5

Nucleotide sequence for hNkd.

GAATTCGCCCTTCTAATACGACTCACTATAGGGCAAGCAGTGGTAACAAC
GCAGAGTACGCGGGGAGTCGGGCCGCGGCGACGGCGGCAGGAGCGC
GTCCCGGCGCCGCCTCGGGCTCCGCTCGGCTCGGGGGCTGCTTCGGG
AGGAGGAGAGCCAAGGGAGGCGCCAGGCCCGCGGGCCGGGCGCATG
GCTTAGGGACGCTCCCGGCCGCCGCAGCCCCAGCATGGGGAAACTTCA
CTCCAAGCCGGCCGCCGTGTGCAAGCGCAGGGAGAGCCCGGAAGGTG
ACAGCTTCGCCGTGAGCGCTGCCTGGGCTCGGAAGGGCATCGAGGAGT
GGATCGGGAGACAGCGCTGCCCGGGCGGTGTCTCGGGACCCCGACAG
CTGCGGTTGGCGGGCACCATAGGCCGAAGCACCCGGGAGCTCGTGGG
CGACGTGTTGAGAGACACGCTCAGCGAGGAAGAGGAGGACGACTTTCG
GCTGGAAGTGGCCCTGCCTCCTGAGAAGACTGACGGGCTGGGCAGCGG
AGATGAGAAGAAGATGGAGAGAGTGAGCGAACCCTGCCCAGGCTCCAA
GAAGCAGCTGAAGTTTGAAGAGCTCCAGTGCGACGTGTCCATGGAGGA
GGACAGCCGGCAGGAGTGGACCTTCACCCTGTATGACTTTGACAACAAC
GGCAAGGTCACCCGAGAGGACATCACCAGCTTGCTGCACACCATCTATG
AGGTGGTGGACTCCTGTGTCAACCACTCCCCAACATCCAGCAAGATGCT
GCGGGTAAAGCTCACCGTGGCCCCCGATGGCAGCCAGAGCAAGAGGAG
CGTCCTTGTCAATCAGGCTGACCTGCAGAGCGCAAGGCCCCGAGCAGA
GACCAAGCCCACTGAGGACCTGCGGAGCTGGGAGAAGAAGCAGCGAGC
CCCGCTCAGGTTCCAGGGTGACAGCCGCCTGGAGCAGTCTGGCTGCTA
CCACCATTGCGTAGATGAGAACATCGAGAGGAGAAACCACTACTTAGAT
CTCGCCGGGATAGAAAACTACACGTCCCAATTTGGGCCTGGCTCCCCTT
CCGTGGCCCAGAAGTCAGAACTGCCCCCCCGCACCTCCAATCCCACTC
GATCTCGCTCCCATGAGCCGGAAGCCATCCACATCCCACACCGAAAGCC
CCAAGGCGTGGACCCGGCCTCCTTCCACTTCCTTGACACCCCAATCGCC
AAGGTCTCAGAGCTCCAGCAACGGCTCCGGGGCACCCAGGACGGGAGC
AAGCACTTTGTGAGGTCCCCCAAGGCCCAGGGCAAGAGTGTGGGTGTG
GGCCACGTGGCCAGAGGGGCAAGAAACAAGCCCCCTCTGGGACCCGCC
ATCCCTGCGGTGTCCCCCTCCGCCCACCTGGCTGCCAGCCCGGCCCTC
CTCCCCTCCCTAGCCCCCCTCGGGCACAAGAAGCACAAGCACCGAGCC
AAGGAGAGCCAGCAGGGCTGCCGGGGCCTGCAGGCACCACTGGCCTC
AGGTGGCCCTGTCCTGGGGCGGGAGCACCTGCGGGAGCTGCCCGCCT
TGGTGGTGTATGAGAGCCAGGCCGGGCAGCCGGTCCAGAGACATGAGC
ACCACCACCACCATGAACATCACCACCATTACCACCACTTCTACCAGACA
TAGAGCCCCTCCCCAGGGCCCCACCCTGCCATATGAAGGACCCCACCC
CCGACACCACAAGGCATTATTATTCTATTAATTATTGTTATTATGATGAT
TTGTTATTAATAATTATTGTTACTCCACTAATATTTAGCTAGCCTACATGT
AGAAGATCTATGGAAACACAGAACTAAACTTTTATTTATATGTTAAAAAAA
AAAAAAAAAAAAAAAAGCGGCCGC

SEQUENCE ID NO: 6

Nucleotide sequence for mNkd.

ATGGGGAAACTTCACTCGAAGCCGGCCGCCGTGTGCAAGCGCAGGGAG
AGC
CCGGAAGGTGACAGCTTTGCTGTAAGCGCTGCTTGGGCAAGGAAAGGC
ATCG
AGGAGTGGATCGGGAGGCAGCGCTGTCCAGGCAGCGTCTCAGGACCCC
GTCA
GCTGAGATTGGCAGGCACTGTTGGTCGAGGCACTCGGGAACTCGTGGG
TGAC
ACTTCTAGAGAGGCTCTCGGTGAGGAGGACGAGGACGACTTCCCCCTA
GAAG
TGGCCCTGCCGCCTGAGAAGATCGACAGCCTAGGTAGTGGAGATGAGA
AGA
GAATGGAGAGACTGAGCGAACCTGGCCAGGCCTCCAAGAAGCAGCTCA
AGT
TTGAAGAGCTACAGTGTGATGTCTCTGTGGAGGAGGACAGCCGGCAAGA
GTG
GACTTTCACTCTATATGACTTCGACAACAATGGCAAAGTGACCCGTGAGG
AC
ATTACCAGCTTGCTGCATACCATCTATGAAGTGGTTGACTCCTCTGTGAA
CCA
TTCCCCCACATCAAGCAAGACACTGCGGGTGAAGCTCACCGTGGCTCCT
GAC
GGGAGCCAGAGTAAGAGGAGCGTCCTTTTCAACCATACCGATCTGCAGA
GCA
CAAGGCCCCGAGCAGACACCAAACCCGCTGAGGAGCTGCGTGGCTGGG
AGA
AGAAGCAGCGAGCCCCACTCAGGTTCCAGGGTGACAGCCACCTGGAGC
AGCC
AGACTGCTACCACCATTGCGTGGATGAGAACATTGAGAGGAGAAACCAC
TAC
CTAGACCTGGCGGGGATAGAGAACTACACGTCTCAGTTTGGACCGGGAT
CCC
CTTCGGTGGCCCAGAAGTCAGAGCTGCCCCCTCGAATCTCCAACCCCAC
TCG
CTCTCGCTCCCACGAGCCAGAAGCTGCCCACATCCCACACCGGAGGCC
CCAA
GGTGTGGACCCAGGCTCCTTCCACCTCCTTGACACCCCATTTGCCAAGG
CATC
AGAGCTCCAGCAACGGCTCCGGGGCACTCAGGATGGGAGCAAGCACTT
TGTG
AGGTCCCCCAAGGCCCAGGGCAAGAACATGGGTATGGGCCACGGGGCC
AGA
GGTGCAAGAAGCAAGCCTCCACTGGTACCCACCACCCATACTGTCTCCC
CCT
CTGCCCATCTGGCCACCAGCCCAGCCCTTCTCCCCACCCTGGCACCCCT
GGGG
CACAAGAAACACAAGCATCGAGCCAAGGAGAGCCAGGCGAGCTGCCGG
GGC
CTGCAGGGCCCCCTGGCTGCAGGAGGCTCCACCGTCATGGGGCGGGA
GCAGG
TGAGGGAGCTGCCTGCCGTGGTGGTGTACGAGAGCCAGGCTGGGCAG
GCCGT
CCAGAGACACGAACACCATCACCACCACCAACATCACCACCATTATCAC
CAC

SEQUENCE ID NO: 7

Amino acid sequence for hNkd.

MGKLHSKPAAVCKRRESPEGDSFAVSAAWARKGIEEWIGRQRCPGGVSGP
RQLRLAGTIGRSTRELVGDVLRDTLSEEEEDDFRLEVALPPEKTDGLGSG
DEKKMERVSEPCPGSKKQLKFEELQCDVSMEEDSRQEWTFTLYDFDNNGKV
TREDITSLLHTIYEVVDSSVNHSPTSSKMLRVKLTVAPDGSQSKRSVLVNQ
ADLQSARPRAETKPTEDLRSWEKKQRAPLRFQGDSRLEQSGCYHHCVDENI
ERRNHYLDLAGIENYTSQFGPGSPSVAQKSELPPRTSNPTRSRSHEPEAIH
IPHRKPQGVDPASFHFLDTPIAKVSELQQRLRGTQDGSKHFVRSPKAQGKS
VGVGHVARGARNKPPLGPAIPAVSPSAHLAASPALLPSLAPLGHKKHKHRA
KESQQGCRGLQAPLASGGPVLGREHLRELPALVVYESQAGQPVQRHEHHH
HHEHHHHYHHFYQT

SEQUENCE ID NO: 8

Amino acid sequence for mNkd.

MGKLHSKPAAVCKRRESPEGDSFAVSAAWARKGIEEWIGRQRCPGSVSGP
RQLR
LAGTVGRGTRELVGDTSREALGEEDEDDFPLEVALPPEKIDSLGSGDEKRM
ERLS
EPGQASKKQLKFEELQCDVSVEEDSRQEWTFTLYDFDNNGKVTREDITSLL
HTIY
EVVDSSVNHSPTSSKTLRVKLTVAPGDSQSKRSVLFNHTDLQSTRPRADTK
PAEE
LRGWEKKQRAPLRFQGDSHLEQPDCYHHCVDENIERRNHYLDLAGIENYTS
QFG
PGSPSVAQKSELPPRISNPTRSRSHEPEAAHIPHRRPQGVDPGSFHLLDTP
FAKASE
LQQRLRGTQDGSKHFVRSPKAQGKNMGMGHGARGARSKPPLVPTTHTVS
PSAH
LATSPALLPTLAPLGHKKHKHRAKESQASCRGLQGPLAAGGSTVMGREQV
RELP
AVVVYESQAGQAVQRHEHHHHHEHHHHYHHFYQP

SEQUENCE ID NO: 9

Nucleotide sequence for putative promoter of hNkd gene.

GAATTCATATGCACATTAAATTCCAGGGAGCCCTCCTCTAGGCTATTTGA
CCCTAGCTCAAGAAAGGGGGATTAAGAGTCTTACAGGGAGGGATCCAAG
GTCAGCATATACAGTTAGTCAGGGACCAGTCTGTCTGTGTCTCTCTCTCC
ATGGGGTTTACTATCATTGCTTTCCCCTAATGGTTCTTACTCCTGCTTCT
TCCTGCTTATTTTTCAGCCCACAGCGACCCCAAGAAGCTGCTCCAACCCC
TGGGACTATGGAGCTCTACAGCTGTAGAGACCACCAGGAAGTGGACTGC
AGGCCCCTGGCCTCTCCATTCAGATTCTGCAAAGAGATCCTGATGGGTT
GGGCCAATGGGTCAGGCATCCAGTCAGCTCTGGCTAAGGGGTGAAGGA
GTCAGGTGTTACCAACGTGGTGGCAGGGGCCACCTTGAAGCTGTGTTCT
GTGCCATGGAAGAAGGAAGAGGAGGAGGAAGCTAAGCTGGAAGGGAAG
GCAGGTGATACAGGAAAATTAACTATGAGCTTTGCTATAGTGACCACTTT
TCCTTCACTCCTTGAGCTGTGGCCTTAAGAACTGTGTACCAATGGGAGG
CACTTGCATAGTAAGTGTTCATTTGCTGAATACTTACAGAGGGCTATAAG
TGGACAAATATGTCCAAAAACACATGAAACACACACCATCAACACTTGCA
GATGGTCTCCTTCAGGGAACCTTTCCACACTGGCTCTCCCCTCACTGAG
CTTTCCTTCCTATCACCCTCCCAGTCTAGGCTCCTGGAGTCAGTAGTTGG
AATCTCAGATGGGAAGAAACCTTAAAAGTCATCTGGTCCAGTATTTTCCA
AAGCATGTTCCATGAACTTGTTTTCCAGAAATGGTTTCCTGGTCTGGTGA
GTTTAAGAAACCCTGCTTATGACGATGCTCTCCATTTAGAGAATCACAAA
GCTTGGCTACTCAATGAAAGCTCTGACAAGTCCTGCAGGAAAAAACTTGT
CTTCTTTTGGCTAAGCTAGGGCTGCCCAAAGTTTCTCATGGAGTCCTTTC
TTGCACATAATAATAGCATCTCACAAACCAGTGGTCGGGGGAACCCATTA
CGGGAAATGCTAATCTTCTGGACCCTTCCTTCTATTTTATAGGTGGAGAG
GCTGTGTGGTGGTCTGGTTGGCTGCATGTAACTGAAAAACAAAGGCTTA
AAAAGATAGGGGCTTCTTTTGCTCTTTTGTTAACAAAGTCTGGGAATAGT
CAAAGACTGGTACTGTGACTAGAAAGGCTTCTGATATGGTTTGGCTCTGT
GTTCCCACCCAAATCTCACCTTGAGTTGTAATGATCCCCATATGTCAAGG
GCAGGATCAGGTGGAGGTAATTGAATCATGAGGGCAGTTAATCCCATGC
TGTTCTTGTGATAGTGAGTTCTCACAGGATCTGATGGTTTTATAAGGGGC
TTTTCCCCTTTGCTCGGCACCTCTCTCTCCTGCTGCTATGTGAAGAGGGA
CGTGTTTGCTTCTCCTTCTGTCATGATTGTAAGTTTCCTGAGGCCTCCCC
AGCCATGTAGAACTGTGAGTCAATTAAACCTCTTTCCTTTATAAATTGCC
CAGTTCCGGTATGTTCTTGTAGCAGCCTGAGAACGGACTAATATAGCTTC
TCTGCCCAGTGTGAAGAAGAGCAGAGAGGCAGGGCTGGGAGGAGAACAA
GGCACCTGCCAAGGAGATGGGGAGGCTGGGCTGGCTTTCCCTCTCCTC
CAGGCTCACCTGGGAAGCCTGTGCTCTAAACTTGCTCAAACATCCTGAA
CCCAGGAGGAGTTGGTGGTACACAAATTCAATTCAATTCAACCCCACATC
CAGACTGTACTCAAGCAGCAGTCTTTTGGCCCAGTCATCTCCAACCTCAT
CTTCTCCCCTCTACTCCCAAACCATGCTCTCTCTGCTCCAGAGCCAGGG
GCCTCTTTGCTGTTTCCAAAACATCCATGGCAGTCTCCACTTCAGGGCCT
TTACATGTGCTGTTCCCTCTGCCTTTAGTACCCAAACAGAATGGCTTGGA
GACCCCAGCCCTAGTTCTTGGGGAAGCCCAGCCTCCTCCATCTCATATC
TAAGGCCTGAGGCCTCCTGGCTGCCTCTGGCTCCCATCTTTTCTCCTGC
AGGGTATCTCCACTGTGAAGATTGCTGTTGGCCCCATTAATTACCTGTAG
GAGTCATCTTTCTGATTCCTTAATTTTGTCCTGTGCCACTAACCCAGGAA
GTGGCTAGGATTTTGCTGAGGGCTTGGAGTAACAGAGGAAGAAGAGAGC
ACTGCCAATCGCATGTTAGAGCTCACTGTCCCAAAAGTGAATTGGCCAGT
CCCCACCTTGCTGGCTGTGCCTTCTTAGGCCCCATTATCACTCTCTTCTC
ATGCTGTTTCCTTTGAGATCTTTGTTTTCCCTTCCCTCCAAAATGCCTGA
TATTTTCTCAGGCAGAGTAGTCTAACTTTCCCCTCCTCACCCAAACTAGG
CTTCCAGGCCCTTTAGCAATGCCAAAACTCACCAGGGAACTATAAAATAA
AGCAACTCCAGGGATCCAGGAGGAACCAGTGGAGACTTGGGAACTGATTT
TCTCCTTCATATATCCAAGATCGTATTATTGGCAGTACTCCCTCCTTATT
GACCACAACATGCTCCTGGCTGAGGCTGGGCAGAGAGAGTTTGTCTCCTT
CCACAGCAGGTGTGATGGCACCTGCTATAGGCAGTGCTGTGTTCGGCAT
TGCATTCAGAGATGCAAGTAAGGCAAGAACCCTGGCCTCAAGGAGCCCT
GGCTCCAGGAGTGAAACTTAGCCTCATGCATAAAATAAGTGGAGATGGG
AGGGGGCAGGGAAACCTTGGCTAATCAGAGCAGAGAAGAGCCCTTCAG
GTTGGAAGGTCAAGGAGGGCTTCCTTTAGGAAATGGCATTTGAAGGGGC
CAGATCAGATGGCTTAACTTCGGGGACAAGCTTTGGAGCAGCTAACTTG
GGTGATGTAGGATTTTTTTTTTTTTTAAATCTCCCAGCTCTATGTCTGAC
AGATTTCACCTAAACCAGCCTTGTTAAATCTCAAGCCCCATGAAACCCGT
TTCTGTTATAATCTCTCTCTCTTATCTCTTCCTTGCCTTCCTTTCCCATT
CTCCTCCCCCAAAGGATAGGAAATCTTCAAAGAAAAAGATGTGTCACTGC
AAGTATACAGCCCAAGAAATGGGCCAGATAAATTATTAAAACACACGAAA
AGACAGTGAGTTATGGGGTGGGAAGCCCTCGGAGGCCGAATGGCCACCCC
AGGTAGACAGCATGCTGGTGGCCCCTGGAGACCCCCTTCTCAGAGACCT
GGACAGACTAACATTTTGCCACAAGGCCCATCTCTTGGGTTCTCACCCCA
GATCTGGGTAAGGGTATCATGATTCCAATAGCAGTGAAGTCCCAGGCGC
CTGCTGGGCTGGGAGGCACCAGGGTGAAGGTGGAGGGGGGCTTTGTGT
CTGGGCTGGACATTTGGGATTTACTCCCCGCAAGACTCAAACTGCTAACT
GCAGTTTTAGTTCTTGCTTTTACCTCTTTTTGAACTGTAAAAAGAAATTC
CCAAGGGGAAGAGGGGATACTTTTTTCTCATGGAAGAAGAAAGCCAGGAC
CGGTTTAAGAAAGTAACCAACTTTCTAAGCACTGTGAGAAAGGATGCTCC
AAGTTTTGCTTTGATTTAGAGGCACCCTGGTACCAGCAGGGAGGGGTGA
GAAAGGCAAACAGGAATTCCAGACGAATTCCATTGCCTTTTGAGGGGTC
TGAAAGAGGGTGCCCACTCCGACTCAGATGCTCAAACCCCTGGCTCCCT
CTTACACCTGACCCCCGCCGTTCTGCCCCACTTTTTCATGTTCCTACAGC
TCAGGGGTTCTTACTTCAGCATTACCCACATTTGATGCTGGATCATTTGT
TCTGGTAGGGTGGGGGGGCTGCCTTATGCATTGTGGTATGTGTAGCAGCA
ACCCTGGCCTCTACCCACTAGATACCTCCAGGCATTACCAAGTGTCCCC
TAGAGGGCAAAATTGTTTGCTGTCAGGTCCTTATGGGATGGAAAGAAAG
AAAAATGGCCTGTTACCCCTGGTGTAACTTACTACACTGTTTACTAATTC
ATCATTTATTGTTTCTTGCCTATCTTCCCCCTAGGTGAGTGGGAGTTCGA
TGAGAGTGGCAGTTGTCTATTTTGTTCACCGATGTATCTTAGGTGACTAA
AACAATGGTTGTCACATGGCTGGCCCTTCATATTTGTTTCCAGATGGAAG
ACTCTCTTTCTAGTGGTGGAACATTAGTTTTGCACTGTGTTGGGACAACC
TGATGTAGTGAAAACAAGCCTGGGCAATGAAATCAACAGATTGGAGTTCA
GTTCCTAATTGGGTCATGGATGAACTTTGTGACCTTGGGCAAGTGAGTTC
ACCTCTCTGAGTTGAATAGGTTCCCTCCTTTCTAGAACAAGTATGAGTCT
GCATCAGAGAGTGGTTGCGAGGGCTACACATGATGGAGGATGAGGACT
GGCACATCAGAAGTACTGAATGAAGAATTGTAACATAAAAATGACAACAG
TAATATATTTTTGTGGTTTCAGCACTCTTCAAATGAAACCACCTGGCCAA
CAGGATTTTAGTGTACCTGCTTATAACATTAGCCTTCGTTTCCACCAAAA
AGGGTGTTAAAAAAGGAAGCTTGGAACATGAAAGTAAGACACTTGGATGA
AGAGATTTATGACTCTGGGGGGCTGTGAATTCCTAATGTCCTTTTGAGAC
ATGTAGATCTTCCAGAGCGATGCTGCCCAATGCAGTAGCCACTAGCCAAG
TGCAAATGGTCACTTGCAATATGGCTAGTCTTTGAGATGTGTTTTAAGTG
TAAAATACACACTGAATTTTAAAGACTTAGCGCAATACAAAGAATGTAAA
ATATCTCATTATATCTTGAAATTATACTATTTTGGATATATGGTGTTCCC
TTGGTGTCTTTGGGGACTGGTTCCAGGATCCTAGAGGATACCCAAATCCC
CAGATGTCAAGTCCGCTATATAAAATGTCCTGTAGTATTTGCATATAACC
TACACACATCCTTCTGAATACTTTAAATCATCTCTAGATTCCTTGTAATT
CCTAATACAATGTAAATGTTATGTAAATAGTTGTTATACTATATTAAAAA
GTTTTTTATTCTTTATTTTTGCTGTATTATTCTTTTTGCATATTTTCAGT
CCACAGATGGTTGATGCCACAGATGTGGAACCTGTGAATAAGGAGGGCTG
ACTGTATTGAGTTAAGCGAAATATATTATTAATATTTCATCTATTTCTTT
TTACTTCTAAAAGATGTGGCGACAAGAAAATTTAAAATTACAAATGTGGC
CCACATTATATTTCTATTGGGCAGTGCTGCTCTAGAGAGTCGGCAAAAAG
GGCAGAATGGAGCCTCCATTATACAGATCACAAAACTGAGCACAGGTAAT
TCACTCCAAAGGTCGGGGCTGGTCTCACTCTGAGCTGCGGGTTTTCTTTT
CCCACGCCAGAGCTGCCTGGTGCCAGGACGAGCGTAACACGGACCCACAG
TGTCCCCAGAAGGGGGCAGGCGTTCTGAGAGCCACAAAGGTGGGGTGGAA
TCCCTTGATGTCGACCGCCACCATCCCCCTCCCCCCGCGCGACCTCCCCG
CAGAGACCTCCCCAGACAAAACAAACAAACCCTTGGGTCTGGCGAACTGC
AGCGGGGAGCGGAAACCAAGGAAGATCAAAGACTCAGCGGTTACCCCCT
TCCGGGCCGCGCAGTTTGGCAGCGCGCCCCGACCCGGGCGGGCACCC
ACGGGCCCCCGGACGAGGAGATCCCAGAGACTGGCTGATAACGGGGC
GCTTTGGACATTTGTCGCTGCCTGGAGAGGGCTGGGCTCACACTGGCC
CGGGGTGCGCTGGGGGCTCCTCCTGGACTCCCCAAATAAGAAACTAGA
GGAGTGCGGTGGTGGGGGGCGGGTCACGGGGCGGGTAATGAACACTT
TCTGCAGAAGGTAGGTCGTGGGAAGACTGGGAAAAGGCAGCGCTGCCG
AAGCTTGCACCTGAGCAGCTAAGGTCTCCGCTCCCGACCTCAGTTTCCC
CACCTGTAAATTGGAGCCGCCGAGTCCCGCCCTGCCCGTTTAGAGAGAA
CGTGGAGCGGAGGGAAGTGACAGTACAGTTAGCGATGGCCGGGCTGTT
CTGTCCCAATACGCCTCCTGGACAAGCCGCCCCGCCGGGTCGCCAGCC
CTGGAGCTCGGCCCCCGGCCCCAGACCGCGGCAGGGAGCGCGGACTG
TGTCCCGCCCCTCCCGTCAGCGCCCCGCCCTCGTCCCCGCCCATGCCC
CGCCTCCGGCCCCGCCCCCGCCGCAACCAGCCTTGCCTTTGATGCGCC
GCACCGGCCAATGGGCGCGCGGGGAGGCGCGGGCCGCGGCGGCGGG
CTGGGGGCTCGGCGCTCCCGGGCGTC

SEQUENCE ID NO: 10

Nucleotide sequence for exon 1: 5′ untranslated region of hNkd gene.

AGTCGGGCCGCGGCGACGGCGGCAGGAGCGCGTCCCGGCGCCGCCTC
GGGCTCCGCTCGGCTCGGGGGCTGCTTCGGGAGGAGGAGAGCCAAGG
GAGGCGCCAGGCCCGCGGGCCGGGCG

SEQUENCE ID NO: 11

Nucleotide sequence for exon 2: 5′ untranslated region of hNkd gene. CATGGCTTAGGGACGCTCCCGGCCGCCGCAGCCCCAGC

SEQUENCE ID NO: 12

Nucleotide sequence for exon 2 (coding) of hNkd gene. ATGGGGAAACTTCACTCCMGCCG

SEQUENCE ID NO: 13

Nucleotide sequence for exon 3 (coding) of hNkd gene. GCCGCCGTGTGCMGCGCAGGGAGAGCCCGGMG

SEQUENCE ID NO: 14

Nucleotide sequence for exon 4 (coding) of hNkd gene.

GTGACAGCTTCGCCGTGAGCGCTGCCTGGGCTCGGAAGGGCATCGAGG
AGTGGATCGGGAGACAGCGCTGCCCGGGCGGTGTCTCGGGACCCCGA
CAGCTGCGGTTGGCGGGCACCATAGGCCGAAGCACCCGG

SEQUENCE ID NO: 15

Nucleotide sequence for exon 5 (coding) of hNkd gene.

GAGCTCGTGGGCGACGTGTTGAGAGACACGCTCAGCGAGGAAGAGGAG
GACGACTTTCGGCTGGAAG

SEQUENCE ID NO: 16

Nucleotide sequence for exon 6 (coding) of hNkd gene.

TGGCCCTGCCTCCTGAGAAGACTGACGGGCTGGGCAGCGGAGATGAGA
AGAAGATGGAGAGAGTGAGCGAACCCTGCCCAGGCTCCAAGAAGCAGC
TGAAGTTTGAA

SEQUENCE ID NO: 17

Nucleotide sequence for exon 7 (coding) of hNkd gene.

GAGCTCCAGTGCGACGTGTCCATGGAGGAGGACAGCCGGCAGGAGTGG
ACCTTCACCCTGTATGACTTTGACAACAACGGCAAGGTCACCCGAGAG

SEQUENCE ID NO: 18

Nucleotide sequence for exon 8 (coding) of hNkd gene.

GACATCACCAGCTTGCTGCACACCATCTATGAGGTGGTGGACTCCTCTG
TCAACCACTCCCCAACATCCAGCAAGATGCTGCGGGTAAAGCTCACCGT
GGCCCCCGATGGCAGCCAGAGCAAGAGGAGCGTCCTTGTCAATCAGGC
TG

SEQUENCE ID NO: 19

Nucleotide sequence for exon 9 (coding) of hNkd gene.

ACCTGCAGAGCGCAAGGCCCCGAGCAGAGACCAAGCCCACTGAGGACC
TGCGGAGCTGGGAGAAGAAGCAGCGAGCCCCGCTCAG

SEQUENCE ID NO: 20

Nucleotide sequence for exon 10 (coding) of hNkd gene.

GTTCCAGGGTGACAGCCGCCTGGAGCAGTCTGGCTGCTACCACCATTG
CGTAGATGAGAACATCGAGAGGAGAAACCACTACTTAGATCTCGCCGGG
ATAGAAAACTACACGTCCCAATTTGGGCCTG

SEQUENCE ID NO: 21

Nucleotide sequence for exon 11 (coding) of hNkd gene.

GCTCCCCTTCCGTGGCCCAGAAGTCAGAACTGCCCCCCCGCACCTCCAA
TCCCACTCGATCTCGCTCCCATGAGCCGGAAGCCATCCACATCCCACAC
CGAAAGCCCCAAGGCGTGGACCCGGCCTCCTTCCACTTCCTTGACACCC
CAATCGCCAAGGTCTCAGAGCTCCAGCAACGGCTCCGGGGCACCCAGG
ACGGGAGCAAGCACTTTGTGAGGTCCCCCAAGGCCCAGGGCAAGAGTG
TGGGTGTGGGCCACGTGGCCAGAGGGGCAAGAAACAAGCCCCCTCTGG
GACCCGCCATCCCTGCGGTGTCCCCCTCCGCCCACCTGGCTGCCAGCC
CGGCCCTCCTCCCCTCCCTAGCCCCCCTCGGGCACAAGAAGCACAAGC
ACCGAGCCAAGGAGAGCCAGCAGGGCTGCCGGGGCCTGCAGGCACCA
CTGGCCTCAGGTGGCCCTGTCCTGGGGCGGGAGCACCTGCGGGAGCT
GCCCGCCTTGGTGGTGTATGAGAGCCAGGCCGGGCAGCCGGTCCAGAG
ACATGAGCACCACCACCACCATGAACATCACCACCATTACCACCACTTCT
ACCAGACATAG

SEQUENCE ID NO: 22

Nucleotide sequence for exon 11 (3′ untranslated region).

AGCCCCTCCCCAGGGCCCCACCCTGCCATATGAAGGACCCCACCCCCG
ACACCACAAGGCATTATTATTCTATTAATTATTGTTATTATGATGATTAT
TGTTATTAATAATTATTGTTACTCCACTAATATTTAGCTAGCCTACATGT
AGAAGATCTATGGAAACACAGAACTAAACTTTTATTTATATGTTAAAAAA
AAAAAAAAAAAAAAAAA

1. An isolated nucleic acid molecule comprising a polynucleotide selected from the group consisting of:
(a) a polynucleotide encoding at least amino acids 2 to 460 of SEQ ID NO:7;
(b) a polynucleotide encoding amino acids 1 to 460 of SEQ ID NO:7
(c) a polynucleotide that encodes at least amino acids 2 to 460 of the human nkd polypeptide having SEQ ID NO:7;
(d) the polynucleotide complement of (a) or (b); or
(e) a polynucleotide that is at least 90% identical to the polynucleotide of (a), (b), (c) or (d).

2. The isolated nucleic acid molecule of claim 1, which contains a polynucleotide encoding at least amino acids 2 to 460 of SEQ ID NO:7.

3. The isolated nucleic acid molecule of claim 1 which contains a polynucleotide encoding amino acids 1 to 460 of SEQ ID NO:7.

4. The isolated nucleic acid molecule of claim 1 which comprises the coding region of SEQ ID NO:5.

5. An isolated nucleic acid molecule which comprises at least 50 contiguous nucleotides of the coding region of SEQ ID NO:5.

6. The isolated nucleic acid molecule of claim 5 which comprises at least 200 contiguous nucleotides of the coding region of SEQ ID NO:5.

7. The isolated nucleic acid molecule of claim 5 which comprises at least 500 contiguous nucleotides of the coding region of SEQ ID NO:5.

8. The isolated nucleic acid molecule of claim 5 which comprises at least 750 contiguous nucleotides of the coding region of SEQ ID NO:5.

9. The isolated nucleic acid molecule of claim 5 which comprises the 1416 nucleotides that constitute the coding region of SEQ ID NO:5.

10. An isolated nucleic acid molecule comprising a nucleic acid sequence that is at least 90% identical to the coding region of SEQ ID NO:5.

11. The isolated nucleic acid molecule of claim 10 which comprises a nucleic acid sequence that is at least 95% identical to the coding region of SEQ ID NO:5.

12. An isolated nucleic acid molecule which encodes a polypeptide that is at least 95% identical to the protein encoded by the coding region of SEQ ID NO:5.

13. An isolated nucleic acid molecule comprising a polynucleotide encoding a polypeptide wherein, except for at least one conservative amino acid substitution, said polypeptide comprises an amino acid sequence selected from the group consisting of
(a) amino acids 2 to 460 of SEQ ID NO:7;
(b) amino acids 1 to 460 of SEQ. ID. NO.: 7;
(c) amino acids 2 to 460 of a non-human primate homologue of SEQ ID NO:7; and
(d) amino acids 1 to 460 of a non-human primate homologue of SEQ ID NO:7.

14. A recombinant vector which comprises an isolated nucleic acid molecule according to claim 1 operably linked to a promoter.

15. A recombinant vector which comprises an isolated nucleic acid molecule according to claim 5 operably linked to a promoter.

16. A recombinant vector which comprises an isolated nucleic acid molecule according to claim 12 operably linked a promoter.

17. A host cell which comprises a recombinant vector according to claim 14.

18. A host cell which comprises a recombinant vector according to claim 15.

19. A host cell which comprises a recombinant vector according to claim 16.

20. A method of producing a polypeptide comprising culturing the host cell of claim 17 under conditions such that said polypeptide is expressed and recovering said polypeptide.

21. A method of producing a polypeptide comprising culturing the host cell of claim 18 under conditions such that said polypeptide is expressed and recovering said polypeptide.

22. A method of producing a polypeptide comprising culturing the host cell of claim 17 under conditions such that said polypeptide is expressed and recovering said polypeptide.

23. An isolated polypeptide comprising amino acids that are at least 95% identical to a polypeptide having the amino acid sequence selected from the group consisting of:
(a) amino acids 1 to 460 of SEQ ID NO:7;
(b) amino acids 2 to 460 of SEQ ID NO:7;
(c) a non-human primate homologue of the polypeptide having SEQ ID NO:7 or a portion thereof comprising at least 460 contiguous amino acids of said homologue.

24. An isolated polypeptide wherein, except for at least one conservative amino acid substitution, said polypeptide has an amino acid sequence selected from the group consisting of:
(a) amino acids 1 to 460 of SEQ ID NO:7;
(b) amino acids 2 to 460 of SEQ ID NO:7;
(c) a non-human primate homologue of the polypeptide corresponding to SEQ ID NO:7 or a portion thereof which comprises at least 460 contiguous amino acids of said homologue.

25. An isolated polypeptide comprising amino acids selected from the group consisting of:
(a) amino acids 1 to 460 of SEQ ID NO:7;
(b) amino acids 2 to 460 of SEQ ID NO:7;
(c) a full length non-human primate homologue of the polypeptide having SEQ ID NO:7 or a portion thereof comprising at least 460 contiguous amino acids of said homologue.

26. A fragment of the polypeptide of SEQ ID NO:7 which elicits the product of an antibody that specifically binds to hNkd but which does not specifically bind murine or Drosophila Nkd.

27. The fragment of claim 25 which comprises at least 50 contiguous amino acids of SEQ ID NO:7.

28. An isolated antibody that specifically binds to the human Nkd polypeptide of SEQ ID NO:7 or a non-human primate homologue thereof but which does not appreciably bind mouse or Drosophila Nkd.

29. The isolated antibody of claim 28-which is a monoclonal antibody.

30. The isolated antibody of claim 29 which is a human, humanized or chimeric monoclonal antibody.

31. A complex comprising a protein having the amino acid sequence of SEQ ID NO:7 or a full-length non-human primate homologue thereof and a Dishevelled protein (Dsh).

32. A complex comprising a fragment of the protein comprising the amino acid sequence shown in SEQ ID NO:7 or a fragment of a non-human primate homologue thereof and a Dishevelled protein (Dsh).

33. The complex of claim 32 wherein said fragment comprises the EF-hand region of the polypeptide shown in SEQ ID NO:7.

34. A method of inhibiting Wnt signaling in a mammalian cell comprising administering the protein having amino acid sequence contained in SEQ ID NO:7 or a full-length non-human primate homologue thereof.

35. The method of claim 34 wherein said mammalian cell is transformed with a vector containing a polynucleotide sequence encoding SEQ ID NO:7.

36. A method of treating a cancer involving aberrant Wnt signaling comprising administrating a therapeutically effective amount of a polypeptide having SEQ ID NO:7, a non-human primate homologue thereof, or a fragment thereof comprising at least 20 contiguous amino acids of SEQ ID NO:7 which elicits the production of an antibody that specifically binds hNkd polypeptide.

37. The method of claim 36 wherein said cancer is selected from the group consisting of colon cancer, head and neck cancer, ovarian cancer and breast cancer.

38. A method of diagnosing a form of cancer potentially involving aberrant Wnt signaling comprising detecting whether human cancer cells obtained from a human patient comprise a human Nkd gene having a coding sequence that differs from the coding sequence having SEQ ID NO:5.
